(19) United States
(12) Patent Application Publication
(10) Pub. No.: US 2017/0165194 A1
(43) Pub. Date: Jun. 15, 2017

(54) COSMETIC AND FACIAL REGENERATION COMPOSITION DERIVED FROM POTENTIATED ADIPOSE DERIVED CELLS AND SUPERNATANTS THEREOF

(71) Applicants: Jiansheng MENG, Xiamen City (CN); Jiong WU, San Diego, CA (US); Feng LIN, San Diego, CA (US)

(72) Inventors: Jiansheng MENG, Xiamen City (CN); Jiong WU, San Diego, CA (US); Feng LIN, San Diego, CA (US)

(21) Appl. No.: 15/379,275

(22) Filed: Dec. 14, 2016

Related U.S. Application Data
(60) Provisional application No. 62/267,197, filed on Dec. 14, 2015.

(51) Int. Cl. A61K 8/98; A61K 8/64; A61Q 19/00

(52) U.S. Cl. CPC A61K 8/981; A61Q 19/00; A61K 8/64; A61K 2800/80

(57) ABSTRACT

This patent discloses new data supporting superior collagen induction by cosmetically useful preparations derived from adipose stem cells that have been manipulated for superior growth factor and anti-aging properties. In one embodiment cellular mixtures derived from adipose tissue are composed, induced to produce regenerative factors, with said regenerative factors harvested and compounded into cosmetic preparations. In one embodiment, the invention provides for manufacture of adipose derived regenerative factor (ARDF), which may be utilized as a cosmetic and skin rejuvenating agent.

### CROSS-REFERENCE TO RELATED APPLICATIONS

This application claims the benefit of priority to U.S. Provisional Application No. 62/267,197, filed, Dec. 14, 2015, which is hereby incorporated in its entirety including all tables, figures, and claims.

### BACKGROUND

The epidermis is the outer layer of the skin, which serves to protect the body against numerous external environment factors such as mechanical traumas, temperature, pathogenic microorganisms, xenobiotics, and UV radiation. Mechanisms exist that act as barriers against factors the action of which accelerate the aging process and skin deterioration. One of these mechanisms is the keratinization process initiated by epidermal keratinocytes that originate from the basal layer through the prickle and granular layer to the horny layer, inside which due to numerous changes they start to transform to korneocytes.

Thus, keratinization means a series of biochemical and morphological changes, being the programmable processes that lead among others, to the cell proliferation ability loss, degeneration of the cell organelle parts and appearing of the new ones, changes in the cell membrane chemical compositions and also appearing of numerous proteins (e.g., involucrine, filagrine binding proteins) and lipids (sterols or phospholipids being the ceramides precursors) (4116).

Researches show that the epidermis granular layer keratinocytes degenerate by apoptosis (3, 7), while the corneocytes located on the horny layer, are subjected to peeling under action of proteolytic enzymes that degrade the corneodesmosomes, i.e., specialized inter-cellular links

present in the epidermis horny layer. The ceramides, being the main inter-cellular cement component, are also subjected to changes: degradation to sphingosine and fat acids, which results in the intercellular cement liquefaction (4). Consequently, the corneocytes are mechanically removed from the epidermis surface.

The epidermis regeneration process is possible thanks to the presence of stem cells in it. Their larger conglomeration is the hair follicle bulge, called the bulge area (3, 8). In this place, such cells intensively divide enlarging its pool, and then they migrate. Part of them migrates to the hair germinal matrix (where they participate in the hair appearing and growth), while the others go to the apical hair part, in order to home the epidermis basal layer.

During the migration to the epidermis, the stem cells meet the area located just over the sebaceous gland, where there are concentrated stem cells responsible for production of sebum (8). However, in order to enable the keratinization and epidermis regeneration process creating the protective layer for the entire organism, many factors must be controlled. Besides the many vitamins, calcium ions, or water level, attention should be paid to a keratinization control process by the keratinocytes themselves. They synthesize and emit many factors that control their process of proliferation and differentiation. They include, among others, EGF, KGF, TGF-alpha, TGF-beta, and IL-1.

Although knowledge exist regarding means of skin self-renewal, means of inducing regeneration of skin in older subjects has been limited. The purpose of the current invention is to provide skin regenerative agents using extracts derived from adipose derived stem cell and other cellular populations.

Utilization of adult stem cells has generated interest in almost all areas of medicine. Studies in heart failure [1-3], liver failure [4-7], stroke [8-10], and wound healing [11, 12], have demonstrated possibility of utilizing mesenchymal stem cells to induce therapeutic benefit. Unfortunately the widespread application of adult stem cells in medicine is still substantially lacking. This is due to lack of products that are approved by regulatory agencies, as well as in situations where such products exist, relatively limited. One of the major revelations in the area of adult stem cell research is that the majority of therapeutic effects are elicited not by MSC directly transdifferentiating into damaged tissue, but by virtue of immune modulation and trophic support to endogenous reparative mechanisms of the host [13-15]. Accordingly, the utilization of stem cell conditioned media/concentration of stem cell derived products has been an area of active investigation [16, 17].

Membrane microvesicles (MMV) are fragments of phospholipid bilayer plasma membrane ranging from 30 nm to 1000 nm shed from almost all cell types. MMV, therefore are a subtype of membrane-vesicles, and play a role in intercellular communication and can deliver mRNA, siRNA, and proteins between cells. They have been implicated in the process of cancer tumour immune suppression, metastasis, tumor-stroma interactions and angiogenesis along with having a role in tissue regeneration. They originate directly from the plasma membrane of the cell and reflect the antigenic content of the cells from which they originate.

In contrast to MMV, exosomes are vesicles of 30-100 nm in diameter, which are actively secreted by a wide range of cell types under both normal and pathological conditions. Exosomes can be regarded as a sub-class of MMV. First discovered in maturing mammalian reticulocytes, they were shown to be a mechanism for selective removal of many plasma membrane proteins and to discard transferrin-receptors from the cell surface of maturing reticulocytes. Although the exosomal protein composition varies with the cell of origin, most exosomes contain the soluble protein Hsc 70 and many others. 31 proteins are found to be in common between colorectal cancer, mast cells and urine-derived exosomes. Certain cells of the immune system, such as dendritic cells and B cells, secrete exosomes that many scientists believe play a functional role in mediating adaptive immune responses to pathogens and tumours.

Exosomes are typically formed through inward budding of endosomal membranes giving rise to intracellular multivesicular bodies (MVB) that later fuse with the plasma membrane, releasing the exosomes to the exterior. In other words, an exosome is created intracellularly when a segment of the cell membrane spontaneously invaginates and is endocytosed. The internalized segment may be broken into smaller vesicles that are subsequently expelled from the cell. The latter stage occurs when the late endosome, containing many small vesicles, fuses with the cell membrane, triggering the release of the vesicles from the cell. The vesicles (once released may be called exosomes) consist of a lipid raft embedded with ligands common to the original cell membrane. However, a more direct release of exosomes has been described, Jurkat T-cells, are said to shed exosomes directly by outward budding of the plasma membrane. Exosomes secreted by cells under normal and pathological conditions contain proteins and functional RNA molecules including mRNA and siRNA, which can be shuttled from one cell to another, affecting the recipient cell's protein production. This RNA is called “exosomal shuttle RNA”. Although exosomes have been previously utilized in the area of regenerative medicine, such as in treatment of heart failure in animal models [18-21], the use of exosomes from adipose tissue has not been previously disclosed for cosmetic and skin regeneration purposes.

### FIELD OF THE INVENTION

The invention belongs to the field of stem cell biology, more specifically, the invention belongs to the field of skin regeneration, more specifically the invention pertains to the use of adipose derived components and secreted factors for the purposes of skin regeneration and rejuvenation.

### BRIEF DESCRIPTION OF THE DRAWINGS

FIG. 1 is a bar graph showing the prevention of dermal fibroblasts apoptosis by ADRF.

FIG. 2 is a bar graph showing ADRF protects Procollagen Type 1 Production from UVA Radiation.

### DESCRIPTION OF THE INVENTION

The invention teaches the use of adipose tissue derived cells and derivatives thereof for production of cosmetics. Adipose tissue is an attractive alternative to bone marrow as a source of regenerative stem cells for the following reasons: a) extraction of adipose derived cells is a simpler procedure that is much less invasive than bone marrow extraction; b) Adipose tissue contains a higher content of mesenchymal stem cells (MSC) as compared to bone marrow; c) MSC from adipose tissue do not decrease in number with aging and can therefore serve as an autologous cell source for all patients; and d) adipose tissue is also a source of unique cell populations in addition to MSC that have therapeutic potential, including endothelial cells and regulatory T cells.

To assist one of skill in the art to practice the disclosed invention, a series of definitions are provided below.

“Anti-inflammatory” means a substance that reduces inflammation. Many analgesics remedy pain by reducing inflammation. Many steroids—specifically glucocorticoids—reduce inflammation by binding to cortisol receptors. Non-steroidal anti-inflammatory drugs (NSAIDs) alleviate pain by counteracting the cyclooxygenase (COX) enzyme. On its own COX enzyme synthesizes prostaglandins, creating inflammation. Many herbs have anti-inflammatory qualities, including but not limited to hyssop and willow bark (the latter of which contains salicylic acid, the active ingredient in aspirin), as well as birch, licorice, wild yam and ginseng. Cytokines such as IL-4, IL-10, TGF-beta, and IL-20 are known to reduce inflammation.

“Antioxidants” means any of a variety of substances that prevent or slow the breakdown of another substance by oxygen. Synthetic and natural antioxidants are used to slow the deterioration of gasoline and rubber, and such antioxidants as vitamin C (ascorbic acid), butylated hydroxytoluene (BHT), and butylated hydroxyanisole (BHA) are typically added to foods to prevent them from becoming rancid or from discoloring. Nutrients such as beta-carotene (a vitamin A precursor), vitellogenin, vitamin C, vitamin E, and selenium have been found to act as antioxidants. They act by scavenging free radicals, molecules with one or more unpaired electrons, which rapidly react with other molecules, starting chain reactions in a process called oxidation. Free radicals are a normal product of metabolism; the body produces its own antioxidants (e.g., the enzyme superoxide dismutase) to keep them in balance. However, stress, aging, and environmental sources such as polluted air and cigarette smoke can add to the number of free radicals in the body, creating an imbalance. The highly reactive free radicals can damage healthy DNA and have been linked to changes that accompany aging (such as age-related macular degeneration, a leading cause of blindness in older people) and with disease processes that lead to cancer, heart disease, and stroke.

An “antiseptic” is a substance that kills or prevents the growth and reproduction of various microorganisms, including bacteria, fungi, protozoa, and viruses on the external surfaces of the body. The objective of antiseptics is to reduce the possibility of sepsis, infection or putrefaction by germs. Antibacterials have the same objective but only act against bacteria. Antibiotics perform a similar function, preventing the growth or reproduction of bacteria within the body. Antiseptics include, but are not limited to, alcohol, iodine, hydrogen peroxide, and boric acid. There is great variation in the ability of antiseptics to destroy microorganisms and in their effect on living tissue. For example, mercuric chloride is a powerful antiseptic, but it irritates delicate tissue. In contrast, silver nitrate kills fewer germs but can be used on the delicate tissues of the eyes and throat. There is also a great difference in the time required for different antiseptics to work. Iodine, one of the fastest-working antiseptics, kills bacteria within 30 sec. Other antiseptics have slower, more residual action. Since so much variability exists, systems have been devised for measuring the action of an antiseptic against certain standards. The bacteriostatic action of an antiseptic compared to that of phenol (under the same conditions and against the same microorganism) is known as its phenol coefficient.

“Chitosan” is a beta-1,4-linked glucosamine polymer which, unlike chitin, contains few, if any, N-acetyl residues. It may be obtained from chitin, a polysaccharide found in the exoskeletons of crustaceans such as shrimp, lobster, and crabs. The shells may be ground into a pulverous powder. This powder is then deacetylated which allows the chitosan to absorb lipids.

“Collagen” means any of a variety of substances that contains the alpha chains of the collagen polypeptide with a sequence that generally follows the pattern Gly-X-Y, where Gly for glycine, X for proline, and Y for proline or hydroxyproline. Collagen proteins also contain significant amounts of glycine and proline. Hydroxyproline and hydroxylysine are not inserted directly by ribosomes. They are derivatised from proline and lysine in enzymatic processes of post-translational modification, for which vitamin C is required. This is related to why vitamin C deficiencies can cause scurvy, a disease that leads to loss of teeth and easy bruising caused by a reduction in strength of connective tissue due to, a lack of collagen, or defective collagen. Cells called fibroblasts form the various fibers in connective tissue in the body including collagen. The white collagen that makes up the matrix of most connective tissue in mammals consists of inter-woven fibres of the protein collagen. The collagen fibers consist of globular units of the collagen sub-unit, tropocollagen. Tropocollagen sub-units spontaneously arrange themselves under physiological conditions into staggered array structures stabilized by numerous hydrogen and covalent bonds. Tropocollagen sub-units are left-handed triple helices where each strand is, further, a right-handed helix itself. Thus, tropocollagen may be considered to be a coiled coil. Although collagen is responsible for skin elasticity, and its degradation leads to wrinkles that accompany aging, it occurs in many other places throughout the body, and in different forms known as types: Type I collagen—This is the most abundant collagen of the human body present in scar tissue, the end product when tissue heals by repair; Type II collagen—Auricular cartilage Type III collagen—This is the collagen of granulation tissue, and is produced quickly by young fibroblasts before the tougher type I collagen is synthesized; Type IV collagen—Basal lamina; Type V collagen—most interstitial tissue, assoc. with type I; Type VI collagen—most interstitial tissue, assoc. with type I; Type VII collagen—epithelia; Type VIII collagen—some endothelial cells; Type IX collagen—cartilage, assoc. with type II; Type X collagen—hypertrophic and mineralizing cartilage; Type XI collagen—cartilage; Type XII collagen—interacts with types I and III.

A “gel” is a semisolid material formed from a colloidal solution. By weight, gels are mostly liquid, yet they behave like solids. An example is gelatin.

The term “natural product” means any of a variety of organic chemical moieties whose molecular arrangement is derived from enzymatic transformations in a living organism excluding amino acids, proteins, polypeptides, nucleic acids and sequences, and saturated fatty acids. Examples include, but are not limited to lipids (i.e., that are not saturated fatty acids), carbohydrates/saccharides and polysaccharides, the steroids and their derivatives, the terpenes and their derivatives, vitamins, carotenoids, and natural medicines such as taxol, etc. The term “synthetic natural product” is a natural product not obtained from its natural source.

The term “gene” as used herein, refers to a DNA sequence that comprises control and coding sequences necessary for the production of a polypeptide or protein precursor. The polypeptide can be encoded by a full length coding sequence or by any portion of the coding sequence, as long as the desired protein activity is retained.

“Nucleoside.” as used herein, refers to a compound consisting of a purine [guanine (G) or adenine (A)] or pyrimidine [thymine (T), uridine (U), or cytidine (C)] base covalently linked to a pentose, whereas “nucleotide” refers to a nucleoside phosphorylated at one of its pentose hydroxyl groups.

“Peptides”, herein defined as polymers formed from the linking, in a defined order, of .alpha.-amino acids; including but not limited to milk peptides, ribosomal peptides, nonribosomal peptides, peptones, cell derived peptides, stem cell derived peptides, immune modulatory peptides and peptide fragments.

“Variant” in regard to amino acid sequences is used to indicate an amino acid sequence that differs by one or more amino acids from another, usually related amino acid. The variant may have “conservative” changes, wherein a substituted amino acid has similar structural or chemical properties (e.g., replacement of leucine with isoleucine). More rarely, a variant may have “non-conservative” changes, e.g., replacement of a glycine with a tryptophan. Similar minor variations may also include amino acid deletions or insertions (i.e., additions), or both. Guidance in determining which and how many amino acid residues may be substituted, inserted or deleted without abolishing biological or immunological activity may be found using computer programs well known in the art, for example, DNAStar software.

“Purified” refers to molecules, including but not limited to nucleic, ribonucleic, lipid or amino acid sequences, which are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

“Cell” means the smallest structural unit of living matter capable of functioning autonomously, consisting of one or more nuclei, cytoplasm, and various organelles, all surrounded by a semipermeable membrane. Cells include all somatic cells obtained or derived from a living or deceased animal body at any stage of development as well as germ cells, including sperm and eggs (animal reproductive body consisting of an ovum or embryo together with nutritive and protective envelopes). Included are both general categories of cells: prokaryotes and eukaryotes. The cells contemplated for use in this invention include all types of cells from all organisms in all kingdoms: plans, animals, protists, fungi, archaebacteria and eubacteria. Stem cells are cells capable, by successive divisions, of producing specialized cells on many different levels. For example, hematopoietic stem cells produce both red blood cells and white blood cells. From conception until death, humans contain stem cells, but in adults their power to differentiate is reduced.

“Differentiation” related to cells means the process by which cells becomes structurally and functionally specialized, which is a progressive restriction of the developmental potential and increasing specialization of function which takes place during the development of the embryo and leads to the formation of specialized cells, tissues, and organs.

“Dedifferentiation” related to cells means the reverse process of differentiation, where cells become less structurally and functionally specialized, which increases the developmental potential of the cell.

“Extract” as used in the context of the current invention means a preparation of any type of cell as defined above obtained by chemical or mechanical action, as by pressure, distillation, evaporation etc. Extracts can include all or any single component or combination of components of the cells, including concentrated preparations of the active components. Such components of the extracts include but are not limited to RNA, DNA, micro RNA, lipids, free amino acids, all amino acid base structures including peptides and proteins, carbohydrates, minerals or combinations thereof.

“Growth media” are compositions used to grow microorganisms or cells in culture. There are different sorts of media for growing different sorts of cells. The biggest difference in growth media are between those used for growing cells in culture (cell culture uses specific cell types derived from plants or animals) and those used for growing microorganisms (usually bacteria or yeast). These differences arise due to the fact that cells derived from whole organisms and grown in culture are often incapable of growth without the provision of certain requirements, such as hormones or growth factors which usually occur in vivo. In the case of animal cells these requirements are often provided by the addition of blood serum to the medium. These media are often red or pink due to the inclusion of pH indicators. Growth media for embryonic stem cells preferably contains minimal essential medium, i.e., Eagle's: amino acids, salts (Ferric nitrate nonahydrate, Potassium chloride, Magnesium sulfate, Sodium chloride, Sodium dihydrogen phosphate), vitamins. (Ascorbic acid, Folic acid, Nicotinamide. Riboflavin, B-12) or Dulbecco's: additionally iron, glucose; non-essential amino acids, sodium pyruvate, .beta.-mercaptoethanol, L-glutamine, fetal bovine serum and Leukemia Inhibitory Factor (LIF). In the case of microorganisms, there are no such limitations as they are often single cell organisms. One other major difference is that animal cells in culture are often grown on a flat surface to which they attach, and the medium is provided in a liquid form, which covers the cells. Bacteria such as Escherichia coli (E. coli, the most commonly used microbe in laboratories) may be grown on solid media or in liquid media, liquid nutrient medium is commonly called nutrient broth. The preferred growth media for microorganisms are nutrient broth or Luria-Bertani medium (L-B medium). Bacterias grown in liquid cultures often form colloidal suspensions. When agar (a substance which sets into a gel) is added to a liquid medium it can be poured into Petri dishes where it will solidify (these are called agar plates) and provide a solid medium on which microbes may be cultured.

“Lipid” means any of a group of organic compounds, including the fats, oils, waxes, sterols, and triglycerides that are insoluble in water but soluble in nonpolar organic solvents, and are oily to the touch. Major classes of lipids include the fatty acids, the glycerol-derived lipids (including the fats and oils and the phospholipids), the sphingosine-derived lipids (including the ceramides, cerebrosides, gangliosides, and sphingomyelins), the steroids and their derivatives, the terpenes and their derivatives, certain aromatic compounds, and long-chain alcohols and waxes. In living organisms lipids serve as the basis of cell membranes and as a form of fuel storage. Often lipids are found conjugated with proteins or carbohydrates, and the resulting substances are known as lipoproteins and lipopolysaccharides. The fat-soluble vitamins can be classified as lipids. Liposomes are spherical vesicles formed by mixing lipids with water or water solutions. They have found applications in the oral administration of some drugs (e.g., insulin and some cancer drugs), since they retain their integrity until they are broken down by the lipases in the stomach and small intestine.

“Prevent” and “preventing” include the prevention of the recurrence, spread or onset. It is not intended that the present invention be limited to complete prevention. In some embodiments, the onset is delayed, or the severity of the disease is reduced.

“Treat” and “treating” are not limited to the case where the subject (e.g. patient) is cured and the disease is eradicated. Rather, the present invention also contemplates treatment that merely reduces symptoms, and/or delays disease progression.

“Transport vehicle, delivery vehicle, and delivery agents” include substances capable of aiding penetration of intact skin or skin cells or other somatic cells. The term “transport vehicle” is used synonymously with the term “permeabilizing agents”. Such transport vehicles include, but are not limited to: phospholipids, palmitylmyristyrates, DMSO, polymer or chitosan suspensions or matrix, liposomes, Trojan peptides, chariot peptides, small elastic vesicles, microspheres (functionalized vectors made from naturally derived materials such as collagen, glycosaminoglycans, chondroitin sulfate, chitosan or polysaccharides), nanoparticles (carries lipophilic substances and enhance bioavailability of the encapsulated material into skin), preloaded spherical beads and sponges, uni- and/or multilamellar vesicles (stabilize contents of extracts in cream base and help transport into skin), retinol molecular film fluid (thin uniform monolayer film that facilitates the transfer of actives through the stratum corneum), poly acrylo nitrile (polymers comprising a controlled release system that synchronizes the release of an active ingredient along with a fragrance as a sensory marker which conveys the efficacy of the product), beta-glucan (oat fiber which aids in penetration of the skin, (Redmond, Int. Journ. Cosmetic science 2005), propylene glycol (as drug carrier, work best with a mineral oil based cream/lotion etc), butylene glycol, polyethylene glycol, olive oil, dimethyl isosorbide, dimethylformamide, methyl salicylate (these all enhance absorption through skin), long chain oleic acids (disrupts the bilayer within the stratum corneum, vital for permeation of compositions in propylene glycol-based formulations), substances capable of adjusting pH, hydration and local metabolism in skin. Agents modifying these factors include a vehicle containing an active hydrophobic agent, de-ionization of active ingredients, increased hydration of the skin (water content of carrier solution/cream/medium), lactic acid (alters the pH).

The invention focuses on cosmetics derived from adipose tissue as a source of regenerative cells. Autologous adipose tissue possesses the advantage of a large number of stem cells being available without need for major in vitro amplification. Additionally, autologous adipose tissue allows for production of individualized cellular products and cellular derived products that are personalized and therefore reducing hypothetical risk of infections. In invention also discloses the use of allogeneic adipose derived stem cells, in this case scalablility and standardization of product is feasible. The safety of adipose derived stem cells is illustrated by numerous clinical trials that have been conducted, which have all demonstrated safety without adverse events. In the practice of the invention potential adverse events associated with stem cell administration include uncontrolled growth, stimulation of carcinogenesis, and ectopic tissue formation. To date, clinical trials on adipose derived cells have utilized ex vivo-expanded cells, which share properties with bone marrow derived MSC [22-27], additionally, several clinical trials have utilized non-expanded stromal vascular fraction (SVF) cells [28-33], which are essentially fat cells devoid of adipocytes, references to these trials are given to provide examples of methodologies used in the art for isolation, purification and expansion of cells useful for the practice of the invention.

Preparations of MSC expanded from adipose tissue are equivalent or superior to bone marrow in terms of differentiation ability [34, 35], angiogenesis-stimulating potential [36], and immune modulatory effects [37]. Given the extra processing steps associated with ex vivo expansion of adipose cells, a simpler and perhaps safer procedure would be the use of primary adipose tissue-derived cells for therapy. SVF comprises the mononuclear cells derived from adipose tissue, which are acquired through a simple isolation procedure whereby fat is lipoaspirated and subjected to enzymatic digestion. In veterinary medicine, over 4,000 horses and 4,000 dogs with various cartilage and bone injuries have been treated with autologous SVF without cellular expansion [38]. In double blind studies of canine osteoarthritis, statistically significant improvements in lameness, range of motion, and overall quality of life have been described [39, 40].

Given the abundance of pre-clinical and veterinary experience with autologous adipose-derived cells, there is great potential for autologous stem cell therapies using adipose-derived cells for a multitude of indications. Indeed, this prospect underlies the interest of commercial entities in devising bench top closed systems for autologous adipose cell therapy, such as Cytori's Celution™ system [41] and Tissue Genesis' TGI1000™ platform [42], which are presently entering clinical trials. Although the majority of studies have focused on in vitro expanded adipose derived cells, SVF derived from whole lipoaspirate alleviates the need for extensive processing of the cells, thereby also minimizing the number of steps where contamination could be introduced. The safety of adipose-derived cells is supported by autologus fat grafting, a common practice in cosmetic surgery [43, 44]. An important consideration in clinical scenerios where bulk SVF is utilized is the potential regenerative, angiogenic and immune regulatory contributions of the numerous cellular populations that are present.

For the purpose of the invention, supernatants are collected from cultured cells derived from adipose tissue, one type of cell, the mesenchymal stem cell, is utilized as a production source of growth factors. Said growth factor production is upregulated in the context of the invention by stimulation in vitro with agents such as cytokines, as well as coculture with cells from adipose tissue that elicit production of cytokines. In one embodiment of the invention coculture of adipose derived mesenchymal stem cells is performed with monocytes. Said monocytes are activated by culture in activatory conditions. In one embodiment monocytes are cultured with TNF-alpha for approximately 1-72 hours, more preferably from 12-48 hours, and more preferably approximately 24 hours. Concentrations of TNF-alpha are approximately 1-1000 ng/million cells, more preferably approximately 10 ng per million cells. Subsequent to culture said monocytes are added to mesenchymal stem cells, in one embodiment at a 1 to 1 ratio, although other ratios are envisioned in the scope of the current invention. Said mesenchymal stem cells and monocyte cultures are allowed to incubate for a period of approximately 72 hours or until reaching confluence. Media useful for the practice of the invention include DMEM, RPMI, AIM-V, OPTI-MEM, and EMEM. Media may be supplemented with nutrients or other factors to maintain viability of cells. Subsequent to culture, said media is harvested and utilized as a component of production of cosmetic. In one embodiment said media may be concentrated by means of lyophilization and subsequent dialysis to remove salt. In one embodiment of the invention, provided is a composition derived from conditioned media of adipose MSC cultured with monocytes, said media useful for formulation into a cosmetic, said media containing one or more factors selected from a group comprising of: a) Interleukin-1 beta; b) Interleukin-6; c) alpha-2-Macroglobulin; d) Midkine; e) Chemokine (C-X-C motif) ligand 1 Chemokine (C-X-C motif) ligands; f) Chemokine (C-X-C motif) ligand 2; g) Chemokine (C-X-C motif) ligand 5; h) Chemokine (C-X-C motif) ligand 6; i) Interleukin-8; j) Chemokine (C-X-C motif) ligand 16; k) Chemokine (C-C motif) ligand 2; l) Chemokine (C-C motif) ligand 8; m) WNT1-inducible-signaling pathway protein 2; n) Fibroblast growth factor 9; o) Platelet-derived growth factor D; p) Vascular endothelial growth factor A; and q) Growth differentiation factor 15. The growth factors/regenerative factors isolated from the conditioned media may be combined with other agents known to be useful in the field of cosmetics, for example liquorice and/or other ingredients that repairs the stratum corneum. Anti-oxidants may be added to the mixture useful for cosmetic purposes. According to the teachings of this invention, one skilled in the art would be aware of various methods for applying the compositions secreted by MSC, MSC-monocyte and manipulated cellular supernatants of this invention to cosmetic products, shampoos, nail strengthening ointments healthcare products and medicaments, and can obtain various desired cosmetic products, healthcare products and medicaments by these methods, so that it is not further described here. Generation of cream or lotion compositions and formulations are described herein, for use in the therapeutic renewal and rejuvenation of the skin of a patient. Specific target components may act, alone or in synergistic combination, to increase the generation of stem, epidermal, or other cells in the skin; to activate or increase collagen synthesis in the skin; to activate or increase endogenous hyaluronic acid synthesis in the epidermis; to activate or increase the hydration of the skin, and to activate or increase the stem cell and fibroblast migration within the epidermis to sites of needed repair on the skin. The use of the skin care composition cream, lotion, or other dermal application compositions of the present invention can yield progress towards dramatically younger looking skin, rehydration and a decrease in signs of aging such as dryness, thin skin, deep wrinkles and dull appearances.

In some embodiments of the invention bone marrow MSC may be utilized in conjunction with adipose derived MSC, or as a substitute, in the preparation of a cosmetic composition that is based on basal secretion of growth factors in conditioned media or stimulated production of growth factors in conditioned media. Specifically, generation of bone marrow derived MSC may be performed as follows. Bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2 [symbol] 10⁷ cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1 [symbol] 10⁶ cells per ml in 175 cm² tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1 [symbol] 10⁶ per 175 cm².

The invention is based on the novel and non-obvious findings that adipose tissue SVF contains cells which when mixed together under stimulatory conditions secrete large amounts of factors capable of inducing skin regeneration. We provide a background on SVF to enable one of skill in the art to practice the invention. SVF was originally described as the proliferative component of adipose tissue by Hollenberg et al. in 1968 [45]. The cells comprising SVF morphologically resemble fibroblasts and were demonstrated to differentiate into pre-adipocytes and functional adipose tissue in vitro [46]. Although it was suggested that non-adipose differentiation of SVF may occur under specific conditions [47], the notion of “adipose-derived stem cells” was not widely recognized until a seminal paper in 2001, where Zuk et al demonstrated the SVF contains large numbers of mesenchymal-like stem cells (MSC-like) cells that could be induced to differentiate into adipogenic, chondrogenic, myogenic, and osteogenic lineages [48]. Subsequent to the initial description, the same group reported that in vitro expanded SVF derived cells had surface marker expression similar to bone marrow derived MSC, displaying expression of CD29, CD44, CD71, CD90, CD105/SH2, and SH3 and lacking CD31, CD34, and CD45 expression [49]. MSC are defined as adherent, non-hematopoietic cells expressing the surface markers CD90, CD105, and CD73, while lacking expression of CD14, CD34, and CD45, and having the ability to differentiate into adipocytes, chondrocytes, and osteocytes in vitro after treatment with the appropriate growth factors [50]. Since these early discoveries research in SVF biological properties has yielded many insights that support the practice of the current invention.

In one embodiment of the invention human lipoaspirates from donors undergoing selective suction-assisted lipectomy is collected. Said liposuctioned aspirate is washed extensively with D-Hanks solution to remove contaminating blood and local anesthetics. The cellular components are subsequently washed two times and plated in T-75 tissue culture flasks at a density of approximately 2×10⁶/ml. Cells are subsequently grown in media containing 57% DMEM/F-12, 40% MCDB-201, 2% fetal calf serum, 10 ng/ml epidermal growth factor, 10 ng/ml platelet-derived growth factor BB, 100 U/ml penicillin, and 100 g/ml streptomycin. Once adherent cells were more than 70% confluent, cells are detached with 0.125% trypsin and 0.01% EDTA, and replated at a 1:3 dilution under the same culture conditions. Cells at 50% adherence are incubated with monocytes derived from adipose tissue, said monocytes are selected by magnetic activated sorting of processed lipoaspirate using positive selection for CD14. Monocytes are activated by exposure to TNF-alpha at a concentration of 10 ng per ml for a culture period of 24 hours. Monocytes are admixed at a 1:1 ratio with cultured MSC for a period of 48 hours. Conditioned media is subsequently extracted and concentrated 25-fold by lyophilization and subsequent desalting. Media is then utilized for formulation into cosmetic solutions.

The invention teaches means of inducing the production of collagen and proliferation of dermal fibroblasts. It is known in the art that the skin is elastic is mainly because the collagen secreted by fibroblast cells in the dermis of the skin, which forms the scaffold of the skin. In one embodiment the cellular conditioned media disclosed in the invention reduces wrinkles by increasing both the number and volume of fibroblast in the skin. The conditioned media disclosed in the invention promotes hydroxyproline synthesis, facilitate the synthesis of collagen and collagenase, secrete collagen as well as, hyaluronic acid and glycoproteins. The media produced in the invention in is generated to specifically stabilize collagenous fibers.

Augmentation of regenerative proteins for the use in the practice of the current invention may be accomplished by transfecting adipose mesenchymal stem cells with genes associated with self-renewal. Said genes are involved in pathways associated with embryonic development (Wnt/β-catenin [51], Notch/Delta-like [52], BMP/SMADs [53]), the hox genes and their partners (Cdx [54], Hoxa9 [55], Hoxa10 [56], Hoxb4 [57], Meis [54], Pbx [54]), and polycomb/trithorax group genes (Bmi1 [58, 59], Ml1 [60]). In addition, a number of transcription factors involved in blood cell differentiation have also been shown to be necessary for self-renewal (Gata-2 [61], Gfi1 [62], JunB [63], Pu.1 [64], Myb [65], Cbp [66], Myc [67], and Zfx [68]). How these diverse pathways are integrated in vivo is not understood; it has been postulated that epigenetic modifications such as chromatin and histone methylation and acetylation play a key role [69], and that the switch between MSC self-renewal and differentiation is regulated by competition between transcription factor complexes, akin to the interplay among Gata-1, c/ebpa, and Pu.1 that mediates the myeloid/erythroid lineage decision [70, 71]. Furthermore, enhancement of self-renewal properties of mesenchymal stem cells from which culture supernatant is collected can be performed by addition of epigenetic modulators. Additionally, epigenetic modulators can be added to the cosmetic formulation itself to enhance efficacy. The present invention is not limited to the use of any particular epigenetic inhibitors. Indeed, the use of variety of epigenetic inhibitors is contemplated, including, but not limited to synthetic epigenetic inhibitors and epigenetic inhibitors isolated or derived from natural sources. Examples of epigenetic inhibitors include, but are not limited to histone deacetylase inhibitors, DNA methyltransferase inhibitors and some vitamins. In some embodiments, the epigenetic inhibitors comprises sodium phenylbutyrate or a natural extract containing butyrate or butyric acid made from natural foods such as butter from animal fats or milk (e.g. cows milk or cheese), plant oils (e.g. Heracleum giganteum (cow parsnip) and Pastinaca sativa (parsnip)), or Kombucha tea (includes Butyric Acid as a result of fermentation containing butyrate). Extract preparation may include fermentation by obligate anaerobic bacteria (e.g. Clostridium butyricum, Clostridium kluyveri. Clostridium pasteurianum, Fusobacterium nucleatum, Butyrivibrio fibrisolvens, Eubacterium limosum). Animal fat or plant oil product extracts may be prepared by chemical or physical processes inducing the liberation of butyric acid from the glyceride by hydrolysis. The extract could also be prepared by the fermentation of sugar or starch in the natural foods by the addition of Bacillus subtilis, with calcium carbonate added to neutralize the acids formed. Epigenetic modulators may be added directly into the tissue culture condition of the MSC, or may be added as part of the formulation that will be applied to the skin. In one embodiment, camphor is added at therapeutic concentrations and frequencies, said therapeutic implying sufficient to stimulate collagen production and inhibit elastase, utilization of camphor as part of cosmetic use is described in the following reference [72], which can be used to guide one of skill in the art in combining camphor to enhance regenerative properties of the invention disclosed herein.

Additionally, cultured MSC may be endowed with certain properties by genetic modification. Since in one embodiment of the invention the active product that is administered to the skin for regeneration is supernatant and not cells, certain genetic modifications to the cells may be performed without risk of modifications being transferred to recipient. In one embodiment MSC are transfected with anti-apoptotic proteins to enhance in vitro longevity. This may be accomplished by transfection with at least one anti-apoptotic protein as a therapy to inhibit or prevent apoptosis. In one embodiment, MSC are contacted with apoptotis cells. In another embodiment, MSC that have been contacted with an apoptotic cell express high levels of anti-apoptotic molecules. In some instances, the MSC that have been contacted with an apoptotic cell secrete high levels of at least one anti-apoptotic protein, including but not limited to, STC-1, BCL-2, XIAP, Survivin, and Bcl-2XL. Methods of transfecting antiapoptotic genes into MSC have been previously described which can be applied to the current invention, said antiapoptotic genes that can be utilized for practice of the invention, in a nonlimiting way, include GATA-4 [73], FGF-2 [74], bc1-2 [75, 76], and HO-1 [77]. Based upon the disclosure provided herein, MSC can be obtained from any source. The MSC may be autologous with respect to the recipient (obtained from the same host) or allogeneic with respect to the recipient. In addition, the MSC may be xenogeneic to the recipient (obtained from an animal of a different species). In one embodiment of the invention MSC are pretreated with agents to induce expression of antiapoptotic genes, one example is pretreatment with exendin-4 as previously described [78]. In a further non-limiting embodiment, MSC used in the present invention can be isolated, from the bone marrow of any species of mammal, including but not limited to, human, mouse, rat, ape, gibbon, bovine. In a non-limiting embodiment, the MSC are isolated from a human, a mouse, or a rat. In another non-limiting embodiment, the MSC are isolated from a human adipose derived MSC.

Based upon the present disclosure, MSC can be isolated and expanded in culture in vitro to obtain sufficient numbers of cells for use in the methods described herein provided that the MSC are cultured in a manner that promotes contact with a tumor endothelial cell. For example, MSC can be isolated from human bone marrow and cultured in complete medium (DMEM low glucose containing 4 mM L-glutamine, 10% FBS, and 1% penicillin/streptomycin) in hanging drops or on non-adherent dishes. The invention, however, should in no way be construed to be limited to any one method of isolating and/or to any culturing medium. Rather, any method of isolating and any culturing medium should be construed to be included in the present invention provided that the MSC are cultured in a manner that provides MSC to express increased amounts of at least one anti-apoptotic protein. Culture conditions for growth of clinical grade MSC have been described in the literature and are incorporated by reference [79-112].

In other embodiments, the epigenetic inhibitors comprise a natural extract of red grapes containing the phytoalexin resveratrol and/or pterostilbene, including an extract from juice or fermented juice (wine) of red grapes. Extracts could be prepared by mechanical disruption of grapes, separation of the skin from the flesh and seeds, and either extracting phytoalexin by chemical or mechanical methods, or be prepared from fresh or fermented grape juice by chemical or physical methods including boiling, fractionation, affinity chromatography, freeze-drying or gel separation.

In other embodiments, the epigenetic inhibitors comprise a natural extract containing Cyanocobalamin (vitamin B-12) made from organisms containing enzymes required for the synthesis of B12 such as bacteria and archaea, or natural products which harbor such B12 producing bacteria including meat (especially liver and shellfish), eggs, and milk products. Extracts can be prepared by chemical or physical methods such as homogenization followed by fractionation, affinity chromatography, freeze-drying or gel separation.

In other embodiments, the epigenetic inhibitors comprise a natural extract containing one or several variants of vitamin B, made from either potatoes, bananas, lentils, chilli peppers, tempeh, liver, turkey, tuna, nutritional yeast (or brewer's yeast), beer or marmite. Extracts can be prepared by chemical or physical methods such as homogenization followed by e.g. fractionation, affinity chromatography, freeze-drying or gel separation. In other embodiments, the epigenetic inhibitors comprise a natural extract containing retinoids or retinoid precursors, made from either animal sources (e.g. milk and eggs) which contain retinyl esters, or from plants (e.g. carrots, spinach) which contain pro-vitamin A carotenoids. The extract may be modified by hydrolysis (animal sources) of retinyl esters to result in retinol, while plant extracts containing pro-vitamin A carotenoids can be cleaved to produce retinal (retinaldehyde), which can be further be reversibly reduced to produce retinol or it can be irreversibly oxidized to produce retinoic acid. The best described active retinoid metabolites are 11-cis-retinal and the all-trans and 9-cis-isomers of retinoic acid, which may be added to this extract.

Examples of other DNA methyltransferase inhibitors include, but are not limited to, 5-Azacytidine. 5-Aza-20-deoxycytidine, Arabinosyl-5-azacytidine, 5-6-Dihydro-5-azacytidine, 5-Fluoro-20-deoxycytidine, EGX30P, Epigallocatechin-3-gallate, Green tea polyphenol, Hydralazine, MG98, Procainamide, Procaine, and Zebularine. Examples of other histone deacetylase inhibitors include, but are not limited to Apicidin, Butyrates. Phenylbutyrate, m-Carboxycinnamic acid bishydroxamide (CBHA). Cyclic hydroxamic-acid-containing peptide 1 (CHAP 1), TSA-Trapoxin Hybrid, Depudecin Epoxide, Depsipeptide FR901228, Benzamidine, LAQ824, Oxamflatin, MGCD0103, PXD101. Pyroxamide, Suberic Bishydroxamic Acid (SBHA), Suberoylanilide Hydroxamic Acid (SAHA), Trichostatin A (TSA), Trapoxin A, and Valproic acid. Other agents that enhance self-renewal may be utilized such as inhibitors of GSK-3, one such inhibitor being lithium. Formulations and use of lithium for stimulation of stem cells are described in the following papers which are incorporated by reference [113-117]. Without being bound to theory, addition of lithium and salts thereof may be incorporated into the cosmetic mixture with the purpose of preventing apoptosis of progenitor cells [118]. Additionally, combinations of epigenetic acting agents together with lithium are envisioned within the practice of the invention to stimulate effects of conditioned media, or to enhance ability of cells to generate conditioned media. Previous combinations of the epigenetic modulator valproic acid with lithium have been published, which can guide one of skill in the art in practice of the invention [119, 120]. Use of lithium to induce dedifferentiation or rejuvenation of cells has previously been performed in experiments in which lithium can enhance inducible pluripotent stem cell generation [121], the generation of these cells being essentially a dedifferentiation of adult stem cells into a pluripotent state.

Agents that can be utilized in the practice of the invention include known cosmetic agents that can be admixed with the cellular conditioned media generated by the invention, said cosmetic agents include botanicals (which may be extracted from one or more of a root, stem bark, leaf, seed or fruit of a plant or plurality of plants). Some botanicals may be extracted from a plant biomass (e.g., root, stem, bark, leaf, etc.) using one more solvents. Botanicals may comprise a complex mixture of compounds and lack a distinct active ingredient. Another category of cosmetic agents that can be admixed with the product of the invention include vitamin compounds and derivatives and combinations thereof, such as a vitamin B3 compound, a vitamin B5 compound, a vitamin B6 compound, a vitamin B9 compound, a vitamin A compound, a vitamin C compound, a vitamin E compound, and derivatives and combinations thereof (e.g., retinol, retinol esters, niacinamide, folic acid, panthenol, ascorbic acid, tocopherol, and tocopherol acetate). Vitamins have been used to prevent or reverse skin damage, and in particular, skin damage associated with inflammation due to UV radiation. Furthermore reference is made to U.S. Pat. Nos. 5,574,063, 5,545,398, 5,409,693, and 5,376,361 which describe the use of fatty acid esters of ascorbic acid (e.g., vitamin C palmitate) or tocotrienol (vitamin E) for treatment and prevention of skin damage. [0093] Anti-oxidants/radical scavengers may be utilized to augment cosmetic effects of the invention, said agents include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the trade name Trolox™), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lysine, pidolate, arginine pidolate, nordihydroguaiaretic acid, bioflavonoids, curcumin, lysine, methionine, proline, Superoxide dismutase, silymarin, tea extracts, grape skin/seed extracts, melanin, and rosemary extracts may be used. Other anti-oxidants/radical scavengers are selected from tocopherol sorbate and other esters of tocopherol. For example, the use of tocopherol sorbate in topical compositions and applicable to the present invention is described in U.S. Pat. No. 4,847,071.

In one embodiment of the invention exosomes are selected from cultures of adipose derived stem cells, or stromal vascular fraction. Said exosomes are purified by anion exchange chromatography. In this way, unexpectedly, it is demonstrated in this application that exosomes are resolved in a homogeneous peak after anion exchange chromatography. This result is completely unexpected given that exosomes are complex supramolecular objects composed, among other things, of a membrane, surrounding an internal volume comprising soluble proteins. In addition, exosomes contain membrane proteins. Therefore, a preferred object of this invention relates to a method of preparing, particularly of purifying, vesicle membranes, from a adipose cell or adipose stem cell conditioned media, comprising at least one anion exchange chromatography step. To apply the invention, a strong or weak, preferably strong, anion exchange may be performed. In addition, in a specific embodiment, the chromatography is performed under pressure. Thus, more specifically, it may consist of high performance liquid chromatography (HPLC). Different types of supports may be used to perform the anion exchange chromatography. More preferably, these may include cellulose, poly(styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: SOURCE. POROS®. SEPHAROSE®, SEPHADEX®, TRISACRYL®, TSK-GEL SW OR PW®, SUPERDEX®TOYOPEARL HW and SEPHACRYL®, for example, which are suitable for the application of this invention.

Therefore, in a specific embodiment, this invention relates to a method of preparing membrane vesicles from a conditioned media, comprising at least one step during which the conditioned media is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalised.

In addition, to improve the chromatographic resolution, within the scope of the invention, it is preferable to use supports in bead form. Ideally, these beads have a homogeneous and calibrated diameter, with a sufficiently high porosity to enable the penetration of the objects under chromatography (i.e. the exosomes). In this way, given the diameter of exosomes (generally between 50 and 100 nm), to apply the invention, it is preferable to use high porosity gels, particularly between 10 nm and 5 .mu.m, more preferably between approximately 20 nm and approximately 2 .mu.m, even more preferably between about 100 nm and about 1 .mu.m.

For the anion exchange chromatography, the support used must be functionalised using a group capable of interacting with an anionic molecule. Generally, this group is composed of an amine which may be ternary or quaternary, which defines a weak or strong anion exchanger, respectively.

Within the scope of this invention, it is particularly advantageous to use a strong anion exchanger. In this way, according to the invention, a chromatography support as described above, functionalised with quaternary amines, is used. Therefore, according to a more specific embodiment of the invention, the anion exchange chromatography is performed on a support functionalised with a quaternary amine. Even more preferably, this support should be selected from poly(styrene-divinylbenzene), acrylamide, agarose, dextran and silica, alone or in mixtures, and functionalised with a quaternary amine.

Examples of supports functionalised with a quaternary amine include the gels SOURCEQ. MONO Q, Q SEPHAROSE®, POROS®HQ and POROS®QE, FRACTOGEL® TMAE type gels and TOYOPEARL SUPER® Q gels.

A particularly preferred support to perform the anion exchange chromatography comprises poly(styrene-divinylbenzene). An example of this type of gel which may be used within the scope of this invention is SOURCE Q gel, particularly SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size.

The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way are detected by measuring their optical density (OD) at the column outlet using a continuous spectro-photometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100 .mu.1 up to 10 ml or greater. In this way, the supports available have a capacity which may reach 25 mg of proteins/ml, for example. For this reason, a 100 .mu.1 column has a capacity of approximately 2.5 mg of proteins which, given the samples in question, allows the treatment of culture supernatants of approximately 2 l (which, after concentration by a factor of 10 to 20, for example, represent volumes of 100 to 200 ml per preparation). It is understood that higher volumes may also be treated, by increasing the volume of the column, for example.

In addition, to perform this invention, it is also possible to combine the anion exchange chromatography step with a gel permeation chromatography step. In this way, according to a specific embodiment of the invention, a gel permeation chromatography step is added to the anion exchange step, either before or after the anion exchange chromatography step. Preferably, in this embodiment, the permeation chromatography step takes place after the anion exchange step. In addition, in a specific variant, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the conditioned media, as described in detail below.

To perform the gel permeation chromatography step, a support selected from silica, acrylamide, agarose, dextran, ethylene glycol-methacrylate co-polymer or mixtures thereof, e.g., agarose-dextran mixtures, are preferably used. As an illustration, for gel permeation chromatography, a support such as SUPERDEX® 200HR (Pharmacia), TSK G6000 (TosoHaas) or SEPHACRYL® S (Pharmacia) is preferably used.

The process according to the invention may be applied to different conditioned medias. In particular, these may consist of a biological fluid from a subject (bone marrow, peripheral blood, etc.), a culture supernatant, a cell lysate, a pre-purified solution or any other composition comprising membrane vesicles.

In this respect, in a specific embodiment of the invention, the conditioned media is a culture supernatant of membrane vesicle-producing cells.

In addition, according to a preferred embodiment of the invention, the conditioned media is treated, prior to the chromatography step, to be enriched with membrane vesicles (enrichment stage). In this way, in a specific embodiment, this invention relates to a method of preparing membrane vesicles from a conditioned media, characterized in that it comprises at least: b) an enrichment step, to prepare a sample enriched with membrane vesicles, and c) a step during which the sample is treated by anion exchange chromatography and/or gel permeation chromatography.

According to a preferred embodiment, the conditioned media is a culture supernatant treated so as to be enriched with membrane vesicles. In particular, the conditioned media may be composed of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography.

Therefore, a preferred method of preparing membrane vesicles according to this invention more particularly comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g. supernatant) enrichment step may comprise one or more centrifugation, clarification, ultrafiltration, nanofiltration and/or affinity chromatography steps on the supernatant. In a first specific embodiment, the enrichment step comprises (i) the elimination of cells and/or cell debris (clarification), possibly followed by (ii) a concentration and/or affinity chromatography step. In an other specific embodiment, the enrichment step comprises an affinity chromatography step, optionally preceded by a step of elimination of cells and/or cell debris (clarification). A preferred enrichment step according to this invention comprises (i) the elimination of cells and/or cell debris (clarification), (ii) a concentration and (iii) an affinity chromatography.

The cells and/or cell debris may be eliminated by centrifugation of the sample, for example, at a low speed, preferably below 1000 g, between 100 and 700 g, for example. Preferred centrifugation conditions during this step are approximately 300 g or 600 g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2 .mu.m, e.g. between 0.2 and 10 .mu.m, are preferentially used. It is particularly possible to use a succession of filters with a porosity of 10 .mu.m, 1 .mu.m, 0.5 .mu.m followed by 0.22 .mu.m.

A concentration step may also be performed, in order to reduce the volumes of sample to be treated during the chromatography stages. In this way, the concentration may be obtained by centrifugation of the sample at high speeds, e.g. between 10,000 and 100,000 g, to cause the sedimentation of the membrane vesicles. This may consist of a series of differential centrifugations, with the last centrifugation performed at approximately 70,000 g. The membrane vesicles in the pellet obtained may be taken up with a smaller volume and in a suitable buffer for the subsequent steps of the process.

The concentration step may also be performed by ultrafiltration. In fact, this ultrafiltration allows both to concentrate the supernatant and perform an initial purification of the vesicles. According to a preferred embodiment, the conditioned media (e.g., the supernatant) is subjected to an ultrafiltration, preferably a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibres (Amicon, Millipore, Sartorius, Pall, GF, Sepracor). Within the scope of the invention, the use of membranes with a cut-off threshold below 1000 kDa, preferably between 300 kDa and 1000 kDa, or even more preferably between 300 kDa and 500 kDa, is advantageous.

The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. Preferably, an affinity chromatography on a dye is used, allowing the elimination (i.e., the retention) of contaminants such as proteins and enzymes, for instance albumin, kinases, deshydrogenases, clotting factors, interferons, lipoproteins, or also co-factors, etc. More preferably, the support used for this chromatography step is a support as used for the ion exchange chromatography, functionalised with a dye. As specific example, the dye may be selected from Blue SEPHAROSE®(Pharmacia), YELLOW 86, GREEN 5 and BROWN 10 (Sigma). The support is more preferably agarose. It should be understood that any other support and/or dye or reactive group allowing the retention (binding) of contaminants from the treated conditioned media can be used in the instant invention.

In a specific embodiment of the invention, the conditioned media is obtained by subjecting a membrane vesicle-producing cell culture supernatant to at least one filtration stage.

In another specific embodiment of the invention, the conditioned media is obtained by subjecting a membrane vesicle-producing cell culture supernatant to at least one centrifugation step.

In a preferred embodiment of the invention, the conditioned media is obtained by subjecting a membrane vesicle-producing cell culture supernatant to at least one ultrafiltration step.

In another preferred embodiment of the invention, the conditioned media is obtained by subjecting a membrane vesicle-producing cell culture supernatant to at least one affinity chromatography step.

A more specific preferred membrane vesicle preparation process within the scope of this invention comprises the following steps: a) the culture of a population of membrane vesicle (e.g. exosome) producing cells under conditions enabling the release of vesicles, b) the treatment of the culture supernatant with at least one ultrafiltration or affinity chromatography step, to produce a conditioned media enriched with membrane vesicles (e.g. with exosomes), and c) an anion exchange chromatography and/or gel permeation chromatography treatment of the conditioned media.

In a preferred embodiment, step b) above comprises a filtration of the culture supernatant, followed by an ultrafiltration, preferably tangential.

In another preferred embodiment, step b) above comprises a clarification of the culture supernatant, followed by an affinity chromatography on dye, preferably on Blue SEPHAROSE®.

In addition, after step c), the material harvested may, if applicable, be subjected to one or more additional treatment and/or filtration stages d), particularly for sterilisation purposes. For this filtration treatment stage, filters with a diameter less than or equal to 0.3 .mu.m are preferentially used, or even more preferentially, less than or equal to 0.25 .mu.m. Such filters have a diameter of 0.22 .mu.m, for example.

After step d), the material obtained is, for example, distributed into suitable devices such as bottles, tubes, bags, syringes, etc., in a suitable storage medium. The purified vesicles obtained in this way may be stored cold, frozen or used extemporaneously.

Therefore, a specific preparation process within the scope of the invention comprises at least the following steps: c) an anion exchange chromatography and/or gel permeation chromatography treatment of the conditioned media, and d) a filtration step, particularly sterilising filtration, of the material harvested after stage c).

In a first variant, the process according to the invention comprises: c) an anion exchange chromatography treatment of the conditioned media, and d) a filtration step, particularly sterilising filtration, on the material harvested after step c).

In another variant, the process according to the invention comprises: c) a gel permeation chromatography treatment of the conditioned media, and d) a filtration step, particularly sterilising filtration, on the material harvested after step c).

According to a third variant, the process according to the invention comprises: c) an anionic exchange treatment of the conditioned media followed or preceded by gel permeation chromatography, and d) a filtration step, particularly sterilising filtration, on the material harvested after step c).

Another means of concentrating exosomes is through their selective affinity to lectins such as GNA, NPA, cyanovirin and ConA. In one particular embodiment of the invention tissue culture media from a culture of adipose derived MSC, alone, or with other cells, is passed through a porous hollow fiber membrane wherein lectin molecules which bind to high mannose glycoproteins are immobilized within the porous exterior portion of the membrane, collecting pass-through tissue culture media and either discarding it, or reusing it. In an embodiment, the method of the present invention is carried out by using an affinity cartridge, with devices of this general type being disclosed in U.S. Pat. Nos. 4,714,556, 4,787,974 and 6,528,057, the disclosures of which are incorporated herein by reference. In this device, tissue culture media from an adipose derived MSC alone or MSC together with other cells is passed through the lumen of a hollow fiber ultrafiltration membrane that is in intimate contact, on the non-blood wetted side of the membrane, with immobilized lectins, which form a means to accept and immobilize viruses and toxic and/or infectious fragments thereof. Thus, the device retains intact exosomes while allowing other components to pass through the lumen. In one embodiment, concentration of exosomes is performed by a device which includes multiple channels of hollow fiber ultrafiltration membrane that forms a filtration chamber. An inlet port and an effluent port are in communication with the filtration chamber. The ultrafiltration membrane is preferably an anisotropic membrane with the tight or retention side facing the bloodstream. The membrane is conveniently formed of any number of polymers known to the art, for example, polysulfone, polyethersulfone, polyamides, polyimides, cellulose acetate, and polyacrylamide. Preferably, the membrane has pores 200 500 nm in diameter, which will allow passage of exosomes and microvesicles. The device in one embodiment comprises a cartridge comprising a tissue culture media-processing chamber formed of interior glass wall. Around chamber is an optional exterior chamber. A temperature controlling fluid can be circulated into chamber through port and out of port. The device includes an inlet port for the tissue culture media and an outlet port for the effluent. The device also provides one or more ports, for accessing the extrachannel space in the cartridge. The technology to immobilize enzymes, chelators, and antibodies in dialysis-like cartridges has been developed (Ambrus et al. Science 201(4358): 837 839, 1978; Ambrus et al. Ann Intern Med 106(4): 531 537, 1987; Kalghatgi et al. Res Commun Chem Pathol Pharmacol 27(3): 551 561, 1980) and is incorporated herein by reference. These cartridges can be directly perfused with tissue culture media from stem cell cultures through direct access to the tissue culture system, and, when desired returned to the tissue culture without further manipulations. Prototypic cartridges have been used to metabolize excess phenylalanine (Kalghatgi et al., 1980, supra; Ambrus, 1978, supra) or to remove excess aluminum from patients' blood (Anthone et al. J Amer Soc Nephrol 6: 1271 1277, 1995). An illustration of preparing proteins for immobilization to the hollow fibers for the method of the present invention is presented in U.S. Pat. Nos. 4,714,556 and 4,787,974, 5,528,057. For binding of lectins to the ultrafiltration membrane, the polymers of the ultrafiltration membrane are first activated, i.e., made susceptible for combining chemically with proteins, by using processes known in the art. Any number of different polymers can be used. To obtain a reactive polyacrylic acid polymer, for example, carbodiimides can be used (Valuev et al., 1998, Biomaterials, 19:41 3). Once the polymer has been activated, the lectins can be attached directly or via a linker to form in either case an affinity matrix. Suitable linkers include, but are not limited to, avidin, strepavidin, biotin, protein A, and protein G. The lectins may also be directly bound to the polymer of the ultrafiltration membrane using coupling agents such as bifunctional reagents, or may be indirectly bound. In a preferred embodiment, GNA covalently coupled to agarose can be used to form an affinity matrix. Once exosomes are bound they may be eluted off using a variety of chemicals known in the art for elution of nanovesicles containing high glycoprotein content from lectins.

In one embodiment of the invention, disclosed is a skin cream comprised of cellular regenerative factors obtained from conditioned media. Skin creams of the invention may comprise several ingredients, some of which are beneficial to the skin, and others that promote absorption of the regeneratively active ingredients into the skin. For example, U.S. Pat. No. 4,362,747 describes a cream pack formulation which comprises a mixture of the following components: (1) propylene glycol and polyoxyethylene; (2) monopalmitate and glyoxyldiureide; (3) alcohol, beeswax, sorbitan monopalmitate, and polyoxyethylene; (4) alcohol, dimethicone copolyol, glyceryl monosterarate/polyoxyethylene; and (5) stearate and zinc or titanium oxide. U.S. Pat. No. 5,391,373 describes a skin cream comprising sodium lactate, a micellar complex of plant extracts, vitamin B, and glycosphingolipids, a protein complex of serum proteins, animal proteins, and glycogen, a carbohydrate based complex of dextran, glycine and glucosamine, a long-chain fatty acid ester of retinol, a long-chain fatty acid ester of ascorbic acid and a short chain fatty acid ester of tocopherol.

Other non-limiting examples of cosmetic agent additives include sugar amines, phytosterols, hexamidine, hydroxy acids, ceramides, amino acids, and polyols. In addition, the generated cosmetics may be admixed with carriers for proper entry of said therapeutic/regenerative molecules into the skin. In the practice of the invention dermatologically acceptable carriers must be safe for use in contact with human skin tissue and not exacerbate existing skin conditions. Suitable carriers that may be contemplated may include water and/or water miscible solvents. The cosmetic skin care composition may comprise from about 1% to about 95% by weight of water and/or water miscible solvent. The composition may comprise from about 1%, 3%, 5%, 10%, 15%, 20%, 25%, 30%, 35%, 40%, 45%, 50%, 55%, 60%, 65%, 70%, 75%, 80%, or 85% to about 90%, 85%, 80%, 75%, 70%, 65%, 60%, 55%, 50%, 45%, 40%, 35%, 30%, 25%, 20%, 15%, 10%, or 5% water and/or water miscible solvents. Examples of water miscible solvents include monohydric alcohols, dihydric alcohols, polyhydric alcohols, glycerol, glycols, polyalkylene glycols such as polyethylene glycol, and mixtures thereof. When the skin care composition is in the form of an emulsion, water and/or water miscible solvents are carriers typically associated with the aqueous phase. Furthermore, carriers may also include oils.

The cosmetic composition may comprise from about 1% to about 95% by weight of one or more oils. The composition may comprise from about 0.1%, 0.5%, 1%, 2%, 5%, 10%, 15%, 20%, 25%, 30%, 35%, 40%, 45%, 50%, 55%, 60%, 65%, 70%, 75%, 80%, 85%, or 90% to about 90%, 85%, 80%, 75%, 70%, 65%, 60%, 55%, 50%, 45%, 40%, 35%, 30%, 25%, 20%, 15%, 10%, 5%, or 3% of one or more oils. Oils may be used to solubilize, disperse, or carry materials that are not suitable for water or water soluble solvents. Suitable oils include silicones, hydrocarbons, esters, amides, ethers, and mixtures thereof. The oils may be volatile or nonvolatile. In one embodiment, silicone oils are utilized as carriers, silicone oils include polysiloxanes. Polysiloxanes that are currently in use include the polydimethylsiloxanes family, which are also known as dimethicones, examples of which include the DM-Fluid series from Shin-Etsu, the Vicasil® series sold by Momentive Performance Materials Inc., and the Dow Corning® 200 series sold by Dow Corning Corporation. Specific examples of suitable polydimethylsiloxanes include Dow Corning® 200 fluids (also sold as Xiameter® PMX-200 Silicone Fluids) having viscosities of 0.65, 1.5, 50, 100, 350, 10,000, 12,500 100,000, and 300,000 centistokes. Other agents that are useful for carrier properties include hydrocarbon oils. Suitable hydrocarbon oils include straight, branched, or cyclic alkanes and alkenes. The chain length may be selected based on desired functional characteristics such as volatility. Suitable volatile hydrocarbons may have between 5-20 carbon atoms or, alternately, between 8-16 carbon atoms. Other suitable oils include esters. The suitable esters typically contained at least 10 carbon atoms. These esters include esters with hydrocarbyl chains derived from fatty acids or alcohols (e.g., mono-esters, polyhydric alcohol esters, and di- and tri-carboxylic acid esters). The hydrocarbyl radicals of the esters hereof may include or have covalently bonded thereto other compatible functionalities, such as amides and alkoxy moieties (e.g., ethoxy or ether linkages, etc.). In another embodiment of the invention amides are utilized as oils. Useful amides include compounds having an amide functional group while being liquid at 20.degree. C. and insoluble in water. Suitable amides include N-acetyl-N-butylaminopropionate, isopropyl N-lauroylsarcosinate, and N,N,-diethyltoluamide. Other suitable amides are disclosed in U.S. Pat. No. 6,872,401. Furthermore, ethers can be utilized for delivery of the invention as carriers. Suitable ethers include saturated and unsaturated fatty ethers of a polyhydric alcohol, and alkoxylated derivatives thereof. Exemplary ethers include C.sub.4-20 alkyl ethers of polypropylene glycols, and di-C.sub.8-30 alkyl ethers. Suitable examples of these materials include PPG-14 butyl ether, PPG-15 stearyl ether, dioctyl ether, dodecyl octyl ether, and mixtures thereof.

Within the purpose of administering the invention, the invented cosmetic may contain an agent capable of emulsifying the cosmetic. The cosmpetic may comprise from about 0.05%, 0.1%, 0.2%, 0.3%, 0.5%, or 1% to about 20%, 10%, 5%, 3%, 2%, or 1% emulsifier. Emulsifiers may be nonionic, anionic or cationic. Non-limiting examples of emulsifiers are disclosed in U.S. Pat. No. 3,755,560, U.S. Pat. No. 4,421,769, and McCutcheon's, Emulsifiers and Detergents, 2010 Annual Ed., published by M. C. Publishing Co. Other suitable emulsifiers are further described in the Personal Care Product Council's International Cosmetic Ingredient Dictionary and Handbook, Thirteenth Edition, 2006, under the functional category of “Surfactants—Emulsifying Agents.” Linear or branched type silicone emulsifiers may also be used. Particularly useful polyether modified silicones include KF-6011, KF-6012, KF-6013, KF-6015, KF-6015, KF-6017, KF-6043, KF-6028, and KF-6038 from Shin Etsu. Also particularly useful are the polyglycerolated linear or branched siloxane emulsifiers including KF-6100, KF-6104, and KF-6105 from Shin Etsu. Emulsifiers also include emulsifying silicone elastomers. Suitable silicone elastomers may be in the powder form, or dispersed or solubilized in solvents such as volatile or nonvolatile silicones, or silicone compatible vehicles such as paraffinic hydrocarbons or esters. Suitable emulsifying silicone elastomers may include at least one polyalkyl ether or polyglycerolated unit.

Viscosity of the cosmetic is particularly important dependent on use or area of the body to be administered. Structuring agents may be used to increase viscosity, thicken, solidify, or provide solid or crystalline structure to the skin care composition. Structuring agents are typically grouped based on solubility, dispersibility, or phase compatibility. Examples of aqueous or water structuring agents include polymeric agents, natural or synthetic gums, polysaccharides, and the like. In one embodiment, the composition may comprises from about 0.0001%, 0.001%, 0.01%, 0.05%, 0.1%, 0.5%, 1%, 2%, 3%, 5% to about 25%, 20%, 10%, 7%, 5%, 4%, or 2%, by weight of the composition, of one or more structuring agents. Furthermore, polysaccharides and gums may be suitable aqueous phase thickening agents. Suitable classes of polymeric structuring agents include but are not limited to carboxylic acid polymers, polyacrylamide polymers, sulfonated polymers, high molecular weight polyalkylglycols or polyglycerins, copolymers thereof, hydrophobically modified derivatives thereof, and mixtures thereof. Silicone gums are another oil phase structuring agent. Another type of oily phase structuring agent includes silicone waxes. Silicone waxes may be referred to as alkyl silicone waxes which and are semi-solids or solids at room temperature. Other oil phase structuring agents may be one or more natural or synthetic waxes such as animal, vegetable, or mineral waxes.

The administration of the cellular regenerative factors/conditioned media may be utilized in conjunction with known approaches in the art to augment efficacy of cosmetic effect. For example, administration by liposomes may be performed as thought in U.S. Pat. No. 6,146,650 which describes the use of liposomes to deliver collagen, avocado oil, aloe and vital nutrients such as Vitamins A, C, D and E to the skin. Also, U.S. Pat. No. 6,281,236 describes cosmetic compositions containing allantoin and an emulsifier such as natural beeswax for the treatment of skin. Utilization of the invention in combination with procedures based on inhibiting existing damaged tissue is disclosed in the current invention, for example, U.S. Pat. No. 6,319,942, teaches the use of alkanolamines such as dimethylaminoethanol (DMAE) for the treatment of scars; U.S. Pat. No. 6,296,861, teaches the use of conjugated linoleic acid and fatty acid esters of vitamin C for treatment of skin damage; U.S. Pat. No. 6,191,121, teaches the use of polyenoylphosphatidyl choline to treat skin damage; U.S. Pat. Nos. 5,965,618 and 5,709,868, teaches the treatment of scar tissue using lipoic acid, and additionally, alpha-hydroxy acids, fatty acid esters of vitamin C, and tocopherol (vitamin E); and U.S. Pat. Nos. 5,554,647 and 5,643,586 teaches the use of catecholamine or acetylcholine precursors for treatment of skin damage.

Envisioned within the current invention is admixing of regenerative factors disclosed in the invention with existing creams and ointments that address underlying skin problems. We incorporate by reference U.S. Pat. Nos. 5,958,397, 5,922,331, 5,817,621, 5,658,580, 5,362,488, 5,322,685, 5,254,331, 4,760,096, 4,297,374, 4,268,526, 4,087,555, and 4,007,266, which all describe the formulation of skin creams which address specific aspects related to skin care. For example, U.S. Pat. No. 5,817,621 describes a skin cream comprising a lipid ointment, vitamin A, a salicylic acid, D-camphor, a biogenic GABAergic substance, a dopaminergic substance, M-cholionolyics, pancreatin, ascorbic acid, pantothenic acid calcium salt, and vitamin D.sub.2 as a means to cause a high trophoprotective effect followed by a restoration of skin physiological functions. Other useful patents for practice of the invention include teaches of U.S. Pat. No. 7,608,642 describes pharmaceutical compositions and methods for managing wound and skin care, in particular methods and compositions that employ compounds that can promote skin cell renewal, wound healing, proliferation of fibroblasts and/or keratinocytes, and the production of collagen.

Adipose stem cells possess numerous therapeutic proteins, however, because they are derived from adult tissue, the anti-aging properties are limited. Accordingly, in one embodiment of the invention the potency of factors secreted from adipose stem cells and combination of adipose stem cells with other cells is increased by pretreatment with therapeutic agents in vitro, prior to isolation and purification of the supernatant. In one specific embodiment valproic acid is added to stimulate production of growth factors by adipose stem cells or cultures of adipose stem cells containing other cells. Means of utilizing valproic acid for stimulation of regenerative properties are known in the literature and one skilled in the art is referred to the following publications for reference [120, 122-131]. Exemplary means of utilizing valproic acid for tissue culture of adipose derived MSC within the practice of the invention includes culture for a period of approximately 48 hours at concentrations of approximately 2.5 mmol/L. The growth factors produced from the cells, combined with the anti-aging factors and subsequently demonstrated to increase collagen synthesis in vitro from fibroblasts, as well as to possess antiaging properties.

### Example 1: Generation of Adipose Stem Cell Derived Regenerative Factor (ADRF)

Adipose stromal vascular fraction cells (SVF) are isolated from human lipoaspirates from donors undergoing selective suction-assisted lipectomy. Said liposuctioned aspirate is washed extensively with D-Hanks solution to remove contaminating blood and local anesthetics. The cellular components are subsequently washed two times in PBS and plated in T-75 tissue culture flasks at a density of approximately 2×10⁶/ml. Cells are subsequently grown in media containing 57% DMEM/F-12, 40% MCDB-201, 2% fetal calf serum, 10 ng/ml epidermal growth factor, 10 ng/ml platelet-derived growth factor BB, 100 U/ml penicillin, and 100 g/ml streptomycin. Once adherent cells were more than 70% confluent, cells are detached with 0.125% trypsin and 0.01% EDTA, and replated at a 1:3 dilution under the same culture conditions. Cells at 50% adherence are incubated with monocytes derived from adipose tissue, said monocytes are selected by magnetic activated sorting of processed lipoaspirate using positive selection for CD14. Monocytes are activated by exposure to TNF-alpha at a concentration of 10 ng per ml for a culture period of 24 hours. Monocytes are admixed at a 1:1 ratio with cultured MSC for a period of 48 hours. Conditioned media is subsequently extracted and concentrated 25-fold by lyophilization and subsequent desalting by dialysis using a dialysis bag possessing a <1 kDa cutoff. The concentrated conditioned media is termed Adipose Derived Regenerative Factor (ADRF). To assess against a control, the same conditions are utilized in the preparation of conditioned media from adipose adherent mesenchymal cells along, without admixing with activated monocytes.

### Example 2: Prevention of Dermal Fibroblast Apoptosis by Adipose Derived Regenerative Factor

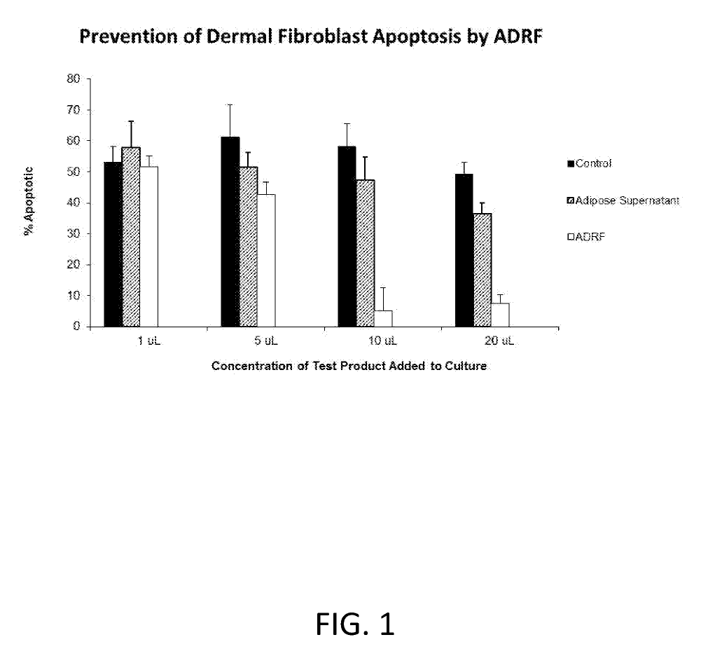

In order to assess whether ADRF prevents apoptosis of fibroblasts, and compare efficacy with control and adipose MSC conditioned media, a standard oxidative stress induced death assay was utilized. H₂O₂ was added to primary dermal fibroblasts to trigger oxidative stress-induced apoptosis, and to assess whether the pretreatment of dermal fibroblasts with test product was able to protect the viability of cells when exposed to oxidative stress. Human dermal fibroblasts were cultured to approximately 80% confluence. Cells were then exposed to products for 30 minutes, products removed and cells treated with 1 mM H₂O₂ for 1 hour. Culture was performed in flat bottom 96 well plates in a total volume of 200 microliters per well with test product added at Concentrations of 1, 5, and 10 uL/well were added of each. Test product consisted of: a) H₂O₂ in the absence of test products; b) adipose conditioned media; and c) ADRF. Concentrations of 1, 5, and 10 uL/well were added of each. H₂O₂ was removed and the cells trypsinized and stained with CellEvent™ Caspase-3/7 Green Flow Cytometry Assay kit (Thermo Fisher Scientific) and acquired by flow cytometry using an Attune® acoustic focusing cytometer. Data was analyzed for either the absence (viable cells) or presence (apoptotic cells) of activated Caspase-3/7, indicated by green fluorescence. See results in FIG. 1.

### Example 3: Stimulation of Collagen Production from Fibroblasts Treated with Adipose Secreted Regenerative Factor

Hs68 cells (human fibroblast cells) were grown in DMEM containing 10% FCS, 0.12% NaHCO₃, penicillin (100 U/mL), streptomycin (100 U/mL), and 5% CO2 in an incubator at 37° C. The T-75 flask was seeded with 1×10⁶ cells, and cells were incubated at 37° C. The cells were harvested at ca. 90% confluence (10⁶ cells/flask), and the survival rates were always higher than 95% by Trypan-blue assay. Cell were then incubated with control DMEM media, Adipose Mesenchymal Supernatant or ADRF for 48 hours. Cells where then transferred in 10-cm² dishes were washed and then covered with 10 mL of Hanks balanced salt solution (1.3 mM CaCl₂, 5.4 mM KCl, 0.4 mM KH₂PO₄, 0.5 mM MgCl₂ 6H₂O, 0.4 mM MgSO₄ 7H₂O, 136.7 mM NaCl, 4.2 mM NaHCO₃ and 0.3 mM NaH₂PO₄ H₂O). Irradiation was carried out in a UVA irradiation chamber (XL-1000 UV cross-linker, Spectronics corporation, Westbury, N.Y., USA) with an accumulated dose of 20 J/cm². The UVA light source emits radiation at a range of 320-380 nm with main output at 365 nm. The surface of the mixture was kept at a distance of 3 cm from the filter surface where the light intensity was 2 mW/cm² s (or 20 W/m² s), as measured using a Vilber Lourmat radiometer (Biotronic UV, Vilber Lourmat, Marne La VallCe, France). After irradiation, the cells were further washed once with PBS, and supplemented with new DMEM, then harvested after 24 h for western blot assay. Sham-irradiated cells were treated in the same manner except that they were not irradiated.

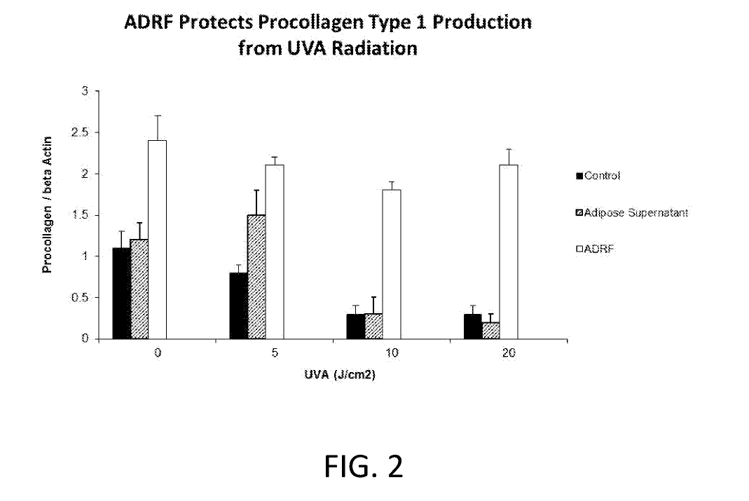

The treated cells were harvested and lysed with 20% SDS containing 1 mM phenylmethylsulfonyl fluoride. The lysate was sonicated for 1 min on ice followed by centrifugation at 12,000 g for 30 min at 4° C. Mitochondrial and cytosolic fractions were isolated by using the ProteoExtract® Cytosol/Mitochondria Fractionation Kit (Merck Millipore, Billerica, Mass., USA). Then a sample of protein from the supernatant was resolved by SDS-PAGE and transferred onto a nitrocellulose membrane. After blocking with TBS buffer (20 mM Tris-HCl, 150 mM NaCl, pH 7.4) containing 5% nonfat milk, the membrane was incubated with antibodies against type I procollagen (Santa Cruz Biotechnology, Santa Cruz, Calif., USA), followed by horseradish peroxidase-conjugated secondary antibodies and then was visualized with an ECL chemiluminescence detection kit (PerkinElmer Life Sciences, Waltham, Mass., USA). The relative density of the immunoreactive bands was quantified by using a luminescent image analyzer (LSA-100, Fujifilm, Japan). See FIG. 2.

### REFERENCES

1. Kim, J., L. Shapiro, and A. Flynn, The clinical application of mesenchymal stem cells and cardiac stem cells as a therapy for cardiovascular disease. Pharmacol Ther, 2015. 151: p. 8-15.
2. Narita, T. and K. Suzuki, Bone marrow-derived mesenchymal stem cells for the treatment of heart failure. Heart Fail Rev, 2015. 20(1): p. 53-68.
3. Choi, Y. H., A. Kurtz, and C. Stamm, Mesenchymal stem cells for cardiac cell therapy. Hum Gene Ther, 2011. 22(1): p. 3-17.
4. Salama, H., et al., Peripheral vein infusion of autologous mesenchymal stem cells in Egyptian HCV-positive patients with end-stage liver disease. Stem Cell Res Ther, 2014. 5(3): p. 70.
5. Shi, M., et al., Human mesenchymal stem cell transfusion is safe and improves liver function in acute-on-chronic liver failure patients. Stem Cells Transl Med, 2012. 1(10): p. 725-31.
6. Amer, M. E., et al., Clinical and laboratory evaluation of patients with end-stage liver cell failure injected with bone marrow-derived hepatocyte-like cells. Eur J Gastroenterol Hepatol, 2011. 23(10): p. 936-41.
7. Saidi, R., et al., Human Adipose-Derived Mesenchymal Stem Cells Promote Liver Regeneration. J Invest Surg, 2015.
8. Dulamea, A. O., The potential use of mesenchymal stem cells in stroke therapy—From bench to bedside. J Neurol Sci, 2015. 352(1-2): p. 1-11.
9. Bhasin, A., et al., Stem cell therapy: a clinical trial of stroke. Clin Neurol Neurosurg, 2013. 115(7): p. 1003-8.
10. Honmou, O., et al., Intravenous administration of auto serum-expanded autologous mesenchymal stem cells in stroke. Brain, 2011. 134(Pt 6): p. 1790-807.
11. Cho, Y. B., et al., Long-term results of adipose-derived stem cell therapy for the treatment of Crohn's fistula. Stem Cells Transl Med, 2015. 4(5): p. 532-7.
12. Wettstein, R., et al., Progenitor cell therapy for sacral pressure sore: a pilot study with a novel human chronic wound model. Stem Cell Res Ther, 2014. 5(1): p. 18.
13. Gallina, C., V. Turinetto, and C. Giachino, A New Paradigm in Cardiac Regeneration: The Mesenchymal Stem Cell Secretome. Stem Cells Int, 2015. 2015: p. 765846.
14. Yang, H. Y., et al., Transplantation of bone marrow mesenchymal stem cells promotes learning and memory functional recovery and reduces hippocampal damage in rats with alcohol-associated dementia. Transplantation, 2015. 99(3): p. 492-9.
15. Madrigal, M., K. S. Rao, and N. H. Riordan, A review of therapeutic effects of mesenchymal stem cell secretions and induction of secretory modification by different culture methods. J Transl Med, 2014. 12: p. 260.
16. Pawitan, J. A., Prospect of stem cell conditioned medium in regenerative medicine. Biomed Res Int, 2014. 2014: p. 965849.
17. Drago, D., et al., The stem cell secretome and its role in brain repair. Biochimie, 2013. 95(12): p. 2271-85.
18. Yu, B., et al., Exosomes secreted from GATA-4 overexpressing mesenchymal stem cells serve as a reservoir of anti-apoptotic microRNAs for cardioprotection. Int J Cardiol, 2015. 182: p. 349-60.
19. Feng, Y., et al., Ischemic preconditioning potentiates the protective effect of stem cells through secretion of exosomes by targeting Mecp2 via miR-22. PLoS One, 2014. 9(2): p. e88685.
20. Arslan, F., et al., Mesenchymal stem cell-derived exosomes increase ATP levels, decrease oxidative stress and activate PI3K/Akt pathway to enhance myocardial viability and prevent adverse remodeling after myocardial ischemia/reperfusion injury. Stem Cell Res, 2013. 10(3): p. 301-12.
21. Barile, L., et al., Ultrastructural evidence of exosome secretion by progenitor cells in adult mouse myocardium and adult human cardiospheres. J Biomed Biotechnol, 2012. 2012: p. 354605.
22. Garcia-Olmo, D., et al., A phase I clinical trial of the treatment of Crohn's fistula by adipose mesenchymal stem cell transplantation. Dis Colon Rectum, 2005. 48(7): p. 1416-23.
23. Stillaert, F. B., et al., Human clinical experience with adipose precursor cells seeded on hyaluronic acid-based spongy scaffolds. Biomaterials, 2008. 29(29): p. 3953-9.
24. Garcia-Olmo, D., M. Garcia-Arranz, and D. Herreros, Expanded adipose-derived stem cells for the treatment of complex perianal fistula including Crohn's disease. Expert Opin Biol Ther, 2008. 8(9): p. 1417-23.
25. Fang, B., et al., Treatment of severe therapy-resistant acute graft-versus-host disease with human adipose tissue-derived mesenchymal stem cells. Bone Marrow Transplant, 2006. 38(5): p. 389-90.
26. Fang, B., et al., Using human adipose tissue-derived mesenchymal stem cells as salvage therapy for hepatic graft-versus-host disease resembling acute hepatitis. Transplant Proc, 2007. 39(5): p. 1710-3.
27. Fang, B., et al., Favorable response to human adipose tissue-derived mesenchymal stem cells in steroid-refractory acute graft-versus-host disease. Transplant Proc, 2007. 39(10): p. 3358-62.
28. Guillaume-Jugnot, P., et al., Autologous adipose-derived stromal vascular fraction in patients with systemic sclerosis: 12-month follow-up. Rheumatology (Oxford), 2015.
29. Kappos, E. A., et al., Peripheral Nerve Repair: Multimodal Comparison of the Long-Term Regenerative Potential of Adipose Tissue-Derived Cells in a Biodegradable Conduit. Stem Cells Dev, 2015. 24(18): p. 2127-41.
30. Gentile, P., et al., A comparative translational study: the combined use of enhanced stromal vascular fraction and platelet-rich plasma improves fat grafting maintenance in breast reconstruction. Stem Cells Transl Med, 2012. 1(4): p. 341-51.
31. Lee, S. K., et al., Facial Soft Tissue Augmentation using Autologous Fat Mixed with Stromal Vascular Fraction. Arch Plast Surg, 2012. 39(5): p. 534-9.
32. Pires de Carvalho, P., et al., Comparison of infrapatellar and subcutaneous adipose tissue stromal vascular fraction and stromal/stem cells in osteoarthritic subjects. J Tissue Eng Regen Med, 2014. 8(10): p. 757-62.
33. Faustini, M., et al., Nonexpanded mesenchymal stem cells for regenerative medicine: yield in stromal vascular fraction from adipose tissues. Tissue Eng Part C Methods, 2010. 16(6): p. 1515-21.
34. Hayashi, O., et al., Comparison of osteogenic ability of rat mesenchymal stem cells from bone marrow, periosteum, and adipose tissue. Calcif Tissue Int, 2008. 82(3): p. 238-47.
35. Noel, D., et al., Cell specific differences between human adipose-derived and mesenchymal-stromal cells despite similar differentiation potentials. Exp Cell Res, 2008. 314(7): p. 1575-84.
36. Kim, Y., et al., Direct comparison of human mesenchymal stem cells derived from adipose tissues and bone marrow in mediating neovascularization in response to vascular ischemia. Cell Physiol Biochem, 2007. 20(6): p. 867-76.
37. Keyser, K. A., K. E. Beagles, and H. P. Kiem, Comparison of mesenchymal stem cells from different tissues to suppress T-cell activation. Cell Transplant, 2007. 16(5): p. 555-62.
38. www.vet-stem.com
39. Black, L. L., et al., Effect of adipose-derived mesenchymal stem and regenerative cells on lameness in dogs with chronic osteoarthritis of the coxofemoral joints: a randomized, double-blinded, multicenter, controlled trial. Vet Ther, 2007. 8(4): p. 272-84.
40. Black, L. L., et al., Effect of intraarticular injection of autologous adipose-derived mesenchymal stem and regenerative cells on clinical signs of chronic osteoarthritis of the elbow joint in dogs. Vet Ther, 2008. 9(3): p. 192-200.
41. Lin, K., et al., Characterization of adipose tissue-derived cells isolated with the Celution system. Cytotherapy, 2008. 10(4): p. 417-26.
42. http://www.tissuegenesis.com/TGI%201000%20Product%20Brochure.pdf.
43. Hang-Fu, L., G. Marmolya, and D. H. Feiglin, Liposuction fat-fillant implant for breast augmentation and reconstruction. Aesthetic Plast Surg, 1995. 19(5): p. 427-37.
44. Klein, A. W., Skin filling. Collagen and other injectables of the skin. Dermatol Clin, 2001. 19(3): p. 491-508, ix.
45. Hollenberg, C. H. and A. Vost, Regulation of DNA synthesis in fat cells and stromal elements from rat adipose tissue. J Clin Invest, 1969. 47(11): p. 2485-98.
46. Gaben-Cogneville, A. M., et al., Differentiation under the control of insulin of rat preadipocytes in primary culture. Isolation of homogeneous cellular fractions by gradient centrifugation. Biochim Biophys Acta, 1983. 762(3): p. 437-44.
47. Glick, J. M. and S. J. Adelman, Established cell lines from rat adipose tissue that secrete lipoprotein lipase. In Vitro, 1983. 19(5): p. 421-8.
48. Zuk, P. A., et al., Multilineage cells from human adipose tissue: implications for cell-based therapies. Tissue Eng, 2001. 7(2): p. 211-28.
49. Zuk, P. A., et al., Human adipose tissue is a source of multipotent stem cells. Mol Biol Cell, 2002. 13(12): p. 4279-95.
50. Prockop, D. J., Marrow stromal cells as stem cells for nonhematopoietic tissues. Science, 1997. 276(5309): p. 71-4.
51. Reya, T., et al., A role for Wnt signalling in self-renewal of haematopoietic stem cells. Nature, 2003. 423(6938): p. 409-14.
52. Stier, S., et al., Notch1 activation increases hematopoietic stem cell self-renewal in vivo and favors lymphoid over myeloid lineage outcome. Blood, 2002. 99(7): p. 2369-78.
53. Karlsson, G., et al., Smad4 is critical for self-renewal of hematopoietic stem cells. J Exp Med, 2007. 204(3): p. 467-74.
54. Schnabel, C. A., Y. Jacobs, and M. L. Cleary, HoxA9-mediated immortalization of myeloid progenitors requires functional interactions with TALE cofactors Pbx and Meis. Oncogene, 2000. 19(5): p. 608-16.
55. Thorsteinsdottir, U., et al., Overexpression of the myeloid leukemia-associated Hoxa9 gene in bone marrow cells induces stem cell expansion. Blood, 2002. 99(1): p. 121-9.
56. Thorsteinsdottir, U., et al., Overexpression of HOXA10 in murine hematopoietic cells perturbs both myeloid and lymphoid differentiation and leads to acute myeloid leukemia. Mol Cell Biol, 1997. 17(1): p. 495-505.
57. Sauvageau, G., et al., Overexpression of HOXB4 in hematopoietic cells causes the selective expansion of more primitive populations in vitro and in vivo. Genes Dev, 1995. 9(14): p. 1753-65.
58. Lessard, J. and G. Sauvageau, Bmi-1 determines the proliferative capacity of normal and leukaemic stem cells. Nature, 2003. 423(6937): p. 255-60.
59. Park, I. K., et al., Bmi-1 is required for maintenance of adult self-renewing haematopoietic stem cells. Nature, 2003. 423(6937): p. 302-5.
60. Ernst, P., et al., Definitive hematopoiesis requires the mixed-lineage leukemia gene. Dev Cell, 2004. 6(3): p. 437-43.
61. Heyworth, C., et al., A GATA-2/estrogen receptor chimera functions as a ligand-dependent negative regulator of self-renewal. Genes Dev, 1999. 13(14): p. 1847-60.
62. Zeng, H., et al., Transcription factor Gfi1 regulates self-renewal and engraftment of hematopoietic stem cells. Embo J, 2004. 23(20): p. 4116-25.
63. Steidl, U., et al., Essential role of Jun family transcription factors in PU.1 knockdown-induced leukemic stem cells. Nat Genet, 2006. 38(11): p. 1269-77.
64. Iwasaki, H., et al., Distinctive and indispensable roles of PU.1 in maintenance of hematopoietic stem cells and their differentiation. Blood, 2005. 106(5): p. 1590-600.
65. White, J. R. and K. Weston, Myb is required for self-renewal in a model system of early hematopoiesis. Oncogene, 2000. 19(9): p. 1196-205.
66. Rebel, V. I., et al., Distinct roles for CREB-binding protein and p300 in hematopoietic stem cell self-renewal. Proc Natl Acad Sci USA, 2002. 99(23): p. 14789-94.
67. Wilson, A., et al., c-Myc controls the balance between hematopoietic stem cell self-renewal and differentiation. Genes Dev, 2004. 18(22): p. 2747-63.
68. Galan-Caridad, J. M., et al., Zfx controls the self-renewal of embryonic and hematopoietic stem cells. Cell, 2007. 129(2): p. 345-57.
69. Zon, L. I., Intrinsic and extrinsic control of haematopoietic stem-cell self-renewal. Nature, 2008. 453(7193): p. 306-13.
70. Nerlov, C. and T. Graf, PU.1 induces myeloid lineage commitment in multipotent hematopoietic progenitors. Genes Dev, 1998. 12(15): p. 2403-12.
71. Nerlov, C., et al., GATA-1 interacts with the myeloid PU.1 transcription factor and represses PU.1-dependent transcription. Blood, 2000. 95(8): p. 2543-51.
72. Tran, T. A., et al., Camphor Induces Proliferative and Anti-senescence Activities in Human Primary Dermal Fibroblasts and Inhibits UV-Induced Wrinkle Formation in Mouse Skin. Phytother Res, 2015.
73. Yu, B., et al., Enhanced mesenchymal stem cell survival induced by GATA-4 overexpression is partially mediated by regulation of the miR-15 family. Int J Biochem Cell Biol, 2013. 45(12): p. 2724-35.
74. Xu, W., et al., Basic fibroblast growth factor expression is implicated in mesenchymal stem cells response to light-induced retinal injury. Cell Mol Neurobiol, 2013. 33(8): p. 1171-9.
75. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.
76. Li, W., et al., Bcl-2 engineered MSCs inhibited apoptosis and improved heart function. Stem Cells, 2007. 25(8): p. 2118-27.
77. Tsubokawa, T., et al., Impact of anti-apoptotic and anti-oxidative effects of bone marrow mesenchymal stem cells with transient overexpression of heme oxygenase-1 on myocardial ischemia. Am J Physiol Heart Circ Physiol, 2010. 298(5): p. H1320-9.
78. Zhou, H., et al., Exendin-4 protects adipose-derived mesenchymal stem cells from apoptosis induced by hydrogen peroxide through the PI3K/Akt-Sfrp2 pathways. Free Radic Biol Med, 2014. 77: p. 363-75.
79. Le Blanc, K., et al., Treatment of severe acute graft-versus-host disease with third party haploidentical mesenchymal stem cells. Lancet, 2004. 363(9419): p. 1439-41.
80. Lazarus, H. M., et al., Cotransplantation of HLA-identical sibling culture-expanded mesenchymal stem cells and hematopoietic stem cells in hematologic malignancy patients. Biol Blood Marrow Transplant, 2005. 11(5): p. 389-98.
81. Bernardo, M. E., et al., Optimization of in vitro expansion of human multipotent mesenchymal stromal cells for cell-therapy approaches: further insights in the search for a fetal calf serum substitute. J Cell Physiol, 2007. 211(1): p. 121-30.
82. Reinisch, A., et al., Humanized system to propagate cord blood-derived multipotent mesenchymal stromal cells for clinical application. Regen Med, 2007. 2(4): p. 371-82.
83. Capelli, C., et al., Human platelet lysate allows expansion and clinical grade production of mesenchymal stromal cells from small samples of bone marrow aspirates or marrow filter washouts. Bone Marrow Transplant, 2007. 40(8): p. 785-91.
84. Lataillade, J. J., et al., New approach to radiation burn treatment by dosimetry-guided surgery combined with autologous mesenchymal stem cell therapy. Regen Med, 2007. 2(5): p. 785-94.
85. Seshareddy, K., D. Troyer, and M. L. Weiss, Method to isolate mesenchymal-like cells from Wharton's Jelly of umbilical cord. Methods Cell Biol, 2008. 86: p. 101-19.
86. Sensebe, L., Clinical grade production of mesenchymal stem cells. Biomed Mater Eng, 2008. 18(1 Suppl): p. S3-10.
87. Sotiropoulou, P. A., S. A. Perez, and M. Papamichail, Clinical grade expansion of human bone marrow mesenchymal stem cells. Methods Mol Biol, 2007. 407: p. 245-63.
88. Shetty, P., et al., Clinical grade mesenchymal stem cells transdifferentiated under xenofree conditions alleviates motor deficiencies in a rat model of Parkinson's disease. Cell Biol Int, 2009. 33(8): p. 830-8.
89. Zhang, X., et al., Cotransplantation of HLA-identical mesenchymal stem cells and hematopoietic stem cells in Chinese patients with hematologic diseases. Int J Lab Hematol, 2010. 32(2): p. 256-64.
90. Arrigoni, E., et al., Isolation, characterization and osteogenic differentiation of adipose-derived stem cells: from small to large animal models. Cell Tissue Res, 2009. 338(3): p. 401-11.
91. Grisendi, G., et al., GMP-manufactured density gradient media for optimized mesenchymal stromal/stem cell isolation and expansion. Cytotherapy, 2010. 12(4): p. 466-77.
92. Prasad, V. K., et al., Efficacy and safety of ex vivo cultured adult human mesenchymal stem cells (Prochymal) in pediatric patients with severe refractory acute graft-versus-host disease in a compassionate use study. Biol Blood Marrow Transplant, 2011. 17(4): p. 534-41.
93. Sensebe, L., P. Bourin, and K. Tarte, Good manufacturing practices production of mesenchymal stem/stromal cells. Hum Gene Ther, 2011. 22(1): p. 19-26.
94. Capelli, C., et al., Minimally manipulated whole human umbilical cord is a rich source of clinical-grade human mesenchymal stromal cells expanded in human platelet lysate. Cytotherapy, 2011. 13(7): p. 786-801.
95. Ilic, N., et al., Manufacture of clinical grade human placenta-derived multipotent mesenchymal stromal cells. Methods Mol Biol, 2011. 698: p. 89-106.
96. Santos, F., et al., Toward a clinical-grade expansion of mesenchymal stem cells from human sources: a microcarrier-based culture system under xeno-free conditions. Tissue Eng Part C Methods, 2011. 17(12): p. 1201-10.
97. Timmins, N. E., et al., Closed system isolation and scalable expansion of human placental mesenchymal stem cells. Biotechnol Bioeng, 2012. 109(7): p. 1817-26.
98. Warnke, P. H., et al., A clinically feasible protocol for using human platelet lysate and mesenchymal stem cells in regenerative therapies. J Craniomaxillofac Surg, 2013. 41(2): p. 153-61.
99. Fekete, N., et al., GMP-compliant isolation and large-scale expansion of bone marrow-derived MSC. PLoS One, 2012. 7(8): p. e43255.
100. Hanley, P. J., et al., Manufacturing mesenchymal stromal cells for phase I clinical trials. Cytotherapy, 2013. 15(4): p. 416-22.
101. Trojahn Kolle, S. F., et al., Pooled human platelet lysate versus fetal bovine serum-investigating the proliferation rate, chromosome stability and angiogenic potential of human adipose tissue-derived stem cells intended for clinical use. Cytotherapy, 2013. 15(9): p. 1086-97.
102. Veronesi, E., et al., Transportation conditions for prompt use of ex vivo expanded and freshly harvested clinical-grade bone marrow mesenchymal stromal/stem cells for bone regeneration. Tissue Eng Part C Methods, 2014. 20(3): p. 239-51.
103. Dolley-Sonneville, P. J., L. E. Romeo, and Z. K. Melkoumian, Synthetic surface for expansion of human mesenchymal stem cells in xeno-free, chemically defined culture conditions. PLoS One, 2013. 8(8): p. e70263.
104. Siciliano, C., et al., Optimization of the isolation and expansion method of human mediastinal-adipose tissue derived mesenchymal stem cells with virally inactivated GMP-grade platelet lysate. Cytotechnology, 2015. 67(1): p. 165-74.
105. Martins, J. P., et al., Towards an advanced therapy medicinal product based on mesenchymal stromal cells isolated from the umbilical cord tissue: quality and safety data. Stem Cell Res Ther, 2014. 5(1): p. 9.
106. Iudicone, P., et al., Pathogen-free, plasma-poor platelet lysate and expansion of human mesenchymal stem cells. J Transl Med, 2014. 12: p. 28.
107. Skrahin, A., et al., Autologous mesenchymal stromal cell infusion as adjunct treatment in patients with multidrug and extensively drug-resistant tuberculosis: an open-label phase 1 safety trial. Lancet Respir Med, 2014. 2(2): p. 108-22.
108. Ikebe, C. and K. Suzuki, Mesenchymal stem cells for regenerative therapy: optimization of cell preparation protocols. Biomed Res Int, 2014. 2014: p. 951512.
109. Chatzistamatiou, T. K., et al., Optimizing isolation culture and freezing methods to preserve Wharton's jelly's mesenchymal stem cell (MSC) properties: an MSC banking protocol validation for the Hellenic Cord Blood Bank. Transfusion, 2014. 54(12): p. 3108-20.
110. Swamynathan, P., et al., Are serum free and xeno-free culture conditions ideal for large scale clinical grade expansion of Wharton's jelly derived mesenchymal stem cells? A comparative study. Stem Cell Res Ther, 2014. 5(4): p. 88.
111. Vaes, B., et al., Culturing protocols for human multipotent adult stem cells. Methods Mol Biol, 2015. 1235: p. 49-58.
112. Devito, L., et al., Wharton's jelly mesenchymal stromal/stem cells derived under chemically defined animal product-free low oxygen conditions are rich in MSCA-1(+) subpopulation. Regen Med, 2014. 9(6): p. 723-32.
113. Dong, B. T., et al., Lithium enhanced cell proliferation and differentiation of mesenchymal stem cells to neural cells in rat spinal cord. Int J Clin Exp Pathol, 2015. 8(3): p. 2473-83.
114. Bernick, J., et al., Parameters for lithium treatment are critical in its enhancement of fracture-healing in rodents. J Bone Joint Surg Am, 2014. 96(23): p. 1990-8.
115. Zhu, Z., et al., Lithium stimulates human bone marrow derived mesenchymal stem cell proliferation through GSK-3beta-dependent beta-catenin/Wnt pathway activation. FEBS J, 2014. 281(23): p. 5371-89.
116. Tsai, H. L., et al., Wnts enhance neurotrophin-induced neuronal differentiation in adult bone-marrow-derived mesenchymal stem cells via canonical and noncanonical signaling pathways. PLoS One, 2014. 9(8): p. e104937.
117. Yoneyama, M., et al., Lithium promotes neuronal repair and ameliorates depression-like behavior following trimethyltin-induced neuronal loss in the dentate gyrus. PLoS One, 2014. 9(2): p. e87953.
118. Cabrera, O., et al., Lithium protects against glucocorticoid induced neural progenitor cell apoptosis in the developing cerebellum. Brain Res, 2014. 1545: p. 54-63.
119. Walasek, M. A., et al., The combination of valproic acid and lithium delays hematopoietic stem/progenitor cell differentiation. Blood, 2012. 119(13): p. 3050-9.
120. Tsai, L. K., et al., Mesenchymal stem cells primed with valproate and lithium robustly migrate to infarcted regions and facilitate recovery in a stroke model. Stroke, 2011. 42(10): p. 2932-9.
121. Wang, Q., et al., Lithium, an anti-psychotic drug, greatly enhances the generation of induced pluripotent stem cells. Cell Res, 2011. 21(10): p. 1424-35.
122. Cho, H. H., et al., Induction of osteogenic differentiation of human mesenchymal stem cells by histone deacetylase inhibitors. J Cell Biochem, 2005. 96(3): p. 533-42.
123. Tsai, L. K., et al., The mood stabilizers valproic acid and lithium enhance mesenchymal stem cell migration via distinct mechanisms. Neuropsychopharmacology, 2010. 35(11): p. 2225-37.
124. Hatakeyama, Y., et al., The effect of valproic Acid on mesenchymal pluripotent cell proliferation and differentiation in extracellular matrices. Drug Target Insights, 2011. 5: p. 1-9.
125. Ryu, C. H., et al., Valproic acid enhances anti-tumor effect of mesenchymal stem cell mediated HSV-TK gene therapy in intracranial glioma. Biochem Biophys Res Commun, 2012. 421(3): p. 585-90.
126. Cho, G. W., et al., Effects of valproic acid on the expression of trophic factors in human bone marrow mesenchymal stromal cells. Neurosci Lett, 2012. 526(2): p. 100-5.
127. Yulin, X., et al., Efficient generation of induced pluripotent stem cells from human bone marrow mesenchymal stem cells. Folia Biol (Praha), 2012. 58(6): p. 221-30.
128. Dong, X., et al., Modification of histone acetylation facilitates hepatic differentiation of human bone marrow mesenchymal stem cells. PLoS One, 2013. 8(5): p. e63405.
129. Jeong, S. G., et al., Valproic acid promotes neuronal differentiation by induction of neuroprogenitors in human bone-marrow mesenchymal stromal cells. Neurosci Lett, 2013. 554: p. 22-7.
130. Marquez-Curtis, L. A., et al., Migration, proliferation, and differentiation of cord blood mesenchymal stromal cells treated with histone deacetylase inhibitor valproic Acid. Stem Cells Int, 2014. 2014: p. 610495.
131. Chen, L., et al., Transplantation of bone marrow mesenchymal stem cells pretreated with valproic acid in rats with an acute spinal cord injury. Biosci Trends, 2014. 8(2): p. 111-9.

1. A composition useful for enhancing skin appearance obtained by the steps of: a) extracting one or more cellular populations from adipose tissue possessing regenerative properties; b) exposing said cells to conditions allowing for production of regenerative factors; c) extracting said regenerative factors; d) concentrating said regenerative factors; e) admixing said regenerative factors in a solution useful for topical application.

2. The composition of claim 1, wherein said cellular population is comprised of cells selected from a group comprising of: a) monocytes; b) T regulatory cells; c) mesenchymal stem cells; and d) endothelial progenitor cells.

3. The composition of claim 2, wherein said cellular populations are expanded ex vivo.

4. The composition of claim 2, wherein said cellular populations are derived from stromal vascular fraction obtained from adipose tissue.

5. The composition of claim 4, wherein said stromal vascular fraction is obtained by the steps of: a) infiltrating an area of the body containing adipose tissue with a solution containing xylocaine and epinephrine; b) extracting adipose tissue in utilizing a cannula attached to a syringe; c) washing said extracted adipose tissue in a saline based solution; d) admixing an enzyme capable of digesting adipose tissue with said washed adipose tissue; e) allowing sufficient time for said enzyme capable of digesting adipose tissue to digest adipose tissue; f) washing said digested adipose tissue so as to pellet the cellular portion of said adipose tissue while discarding remains of said adipose tissue.

6. The composition of claim 2, wherein said monocytes are collected from said adipose tissue by means of plastic adherence.

7. The composition of claim 2, wherein said monocytes are collected from said adipose tissue by means of Magnetic Activated Cell Sorting (MACS).

8. The composition of claim 7, wherein said selection of monocytes by MACS is achieved by use of antibody targeting CD14.

9. The composition of claim 2, wherein said T regulatory cells are isolated from said adipose tissue by means of MACS.

10. The composition of claim 9, wherein said selection of T regulatory cells by MACS is achieved by use of antibody targeting CD25.

11. The composition of claim 2, wherein said mesenchymal stem cells are isolated from adipose tissue by means of plastic adherence.

12. The composition of claim 2, wherein said mesenchymal stem cells are isolated from adipose tissue by means of growth in mesenchymal stem cell promoting media.

13. The composition of claim 2, wherein said mesenchymal stem cells are isolated from adipose tissue by means of MACS.

14. The composition of claim 2, wherein said mesenchymal stem cells are isolated from adipose tissue by means of MACS using antibody targeting STRO-1.

15. The composition of claim 1, wherein said regenerative factors are concentrated by lyophilization.

16. The composition of claim 1, wherein said regenerative factors are exosomes.

17. The composition of claim 16, wherein said exosomes are concentrated by an affinity means.

18. The composition of claim 17, wherein said affinity means is column chromatography.

19. The composition of claim 17, wherein said affinity means involves exposing conditioned media through a column containing agents with selective affinity to exosomes.

20. The composition of claim 19, wherein said agents with selective affinity to exosomes are selected from a group comprising of: a) a protein; b) an antibody; c) a DNano particle; d) a lectin; and e) an aptamer.

21. The composition of claim 19, wherein said lectin is selected from a group comprising of: a) Galanthus nivalis agglutinin (GNA), b) Narcissus pseudonarcissus agglutinin (NPA), c) cyanovirin and d) Conconavalin A.

22. The composition of claim 16, wherein said exosomes possess the following characteristics: (a) have a size of between 50 nm and 100 nm as determined by electron microscopy; (b) comprises a complex of molecular weight >100 kDa, for example comprising proteins of <100 kDa; (c) comprises a complex of molecular weight >300 kDa, for example comprising proteins of <300 kDa; (d) comprises a complex of molecular weight >1000 kDa; (e) has a size of between 2 nm and 200 nm, such as a size of between 50 nm and 150 nm or a size of between 50 nm and 100 nm, for example as determined by filtration against a 0.2 .mu.M filter and concentration against a membrane with a molecular weight cut-off of 10 kDa; or (f) a hydrodynamic radius of below 100 nm, such as between about 30 nm and about 70 nm, between about 40 nm and about 60 nm, such as between about 45 nm and about 55 nm, such as about 50 nm, for example as determined by laser diffraction or dynamic light scattering.

23. The composition of claim 1, wherein said cells derived from adipose tissue are exposed to conditions selected from a group comprising of: a) hypoxia; b) hyperthermia; c) hypotonic challenge; d) oxidative stress; and e) inflammatory stimuli.

24. The composition of claim 23, wherein said exposure to said conditions is performed to augment production of regenerative factors.

25. The composition of claim 1, wherein said composition is admixed with platelet rich plasma.

26. The composition of claim 1, wherein human chorionic gonadotropin is admixed with said composition.

27. The composition of claim 1, wherein one or more antioxidants are added to said composition.

28. The composition of claim 1, wherein one or more antiseptic agents are added to said composition.

29. The composition of claim 1, wherein one or more anti-inflammatory agents are added to said composition.

30. The composition of claim 1, wherein one more delivery vehicles are admixed with said composition.

31. The composition of claim 1, wherein said delivery vehicle is selected from a group comprising of: a) phospholipids; b) palmitylmyristyrates; c) DMSO; d) a polymer or chitosan suspensions or matrix; d) liposomes; e) Trojan peptides; f) chariot peptides; g) small elastic vesicles; h) microspheres.

32. The composition of claim 31, wherein said microspheres are made from naturally derived materials selected from a group comprising of: a) collagen; b) glycosaminoglycans; c) chondroitin sulfate and d) chitosan or polysaccharides.

33. The composition of claim 1, wherein said composition is administered together with a nanoparticle delivery vehicle capable of transferring the epidermis.

34. The composition of claim 1, wherein said composition is admixed with agents selected from a group comprising of: a) beta-glucan; b) propylene glycol; c) butylene glycol; d) polyethylene glycol; e) olive oil; f) dimethyl isosorbide; g) dimethylformamide; h) methyl salicylate; i) long chain oleic acids; and j) lactic acid.
